(19) United States
(12) Patent Application Publication
(10) Pub. No.: US 2023/0416457 A1
(43) Pub. Date: Dec. 28, 2023

(54) DUAL MODALITY UPS NANOPROBES FOR TUMOR ACIDOSIS IMAGING

(71) Applicant: The Board of Regents of The University of Texas System, Austin, TX (US)

(72) Inventors: Jinming GAO, Plano, TX (US); Gang HUANG, Plano, TX (US); Tian ZHAO, Irving, TX (US); Baran D. SUMER, Dallas, TX (US); Xiankai SUN, Coppell, TX (US)

(73) Assignee: The Board of Regents of The University of Texas System, Austin, TX (US)

(21) Appl. No.: 18/159,084

(22) Filed: Jan. 24, 2023

Related U.S. Application Data
(63) Continuation of application No. 16/570,337, filed on Sep. 13, 2019, now abandoned.
(60) Provisional application No. 62/731,848, filed on Sep. 15, 2018.

(51) Int. Cl. C08G 65/00; A61K 49/00; G01N 33/58; G01N 21/64; G01N 33/84

(52) U.S. Cl. CPC C08G 65/007; A61K 49/0054; G01N 33/582; G01N 21/643; G01N 21/6456; G01N 33/84; G01N 2021/6441

(57) ABSTRACT

The present disclosure relates to polymers which contain a hydrophobic and hydrophilic segment which is sensitive to pH as well as a metal chelating group. In some aspects, the metal chelating group is chelated to a metal ion capable of positron emission. In some aspects, the polymers form a micelle which is sensitive to pH and results in a change in fluorescence based upon the particular pH. In some aspects, the disclosure also provides methods of using the polymers for the imaging of cellular or extracellular environment or delivering a drug.

### PRIORITY CLAIM

This application is a continuation of U.S. application Ser. No. 16/570,337, filed Sep. 13, 2019, which claims benefit of priority to U.S. Provisional Application Ser. No. 62/731,848, filed Sep. 15, 2018, the entire contents of each of which are hereby incorporated by reference.

This invention was made with government support under Grant Number R01 CA192221 awarded by the National Institutes of Health. The government has certain rights in the invention.

### BACKGROUND

### 1. Field

The present disclosure relates generally to the fields of molecular and cellular biology, cancer imaging, nanotechnology, fluorescence sensors, and sensors for positron emission topography. More particularly, it relates to nanoplatforms for the detection of pH changes.

### 2. Description of Related Art

Cancer exhibits diverse genetic and histological differences from normal tissues (Vogelstein et al., 2013). Molecular characterization of these differences is useful to stratify patients towards personalized therapy. However, the strategy may not serve as a broad diagnostic tool because genetic/phenotypic biomarkers are expressed in a subset of patients and significant overlap with normal tissues exist (Jacobs et al., 2000 and Paik et al., 2000). Deregulated energetics is a hallmark of cancer that occurs across many types of cancer (Hanahan and Weinberg, 2011). Elevated glucose metabolism in cancer cells has long been associated with aerobic glycolysis, where cancer cells preferentially take up glucose and convert it into lactic acid (Heiden et al., 2009). More recent studies using ¹³C-labelled glucose in lung cancer patients further demonstrate accelerated oxidative phosphorylation in addition to glycolysis as a cancer cell mechanism for growth and proliferation (Hensley et al., 2016). The clinical significance of the glucose metabolism is manifested by the wide use of ¹⁸F-fluorodeoxyglucose (FDG) positron emission tomography (PET; Zhu et al., 2011) where FDG, a radiolabeled glucose analog, is selectively taken up by overexpressed glucose transporters and trapped inside the cancer cells after phosphorylation by hexokinase for PET detection (Som et al., 1980).

Despite broad clinical adoption, FDG has many well-described pitfalls (Cook et al., 2004, Purohit et al., 2014, Truong et al., 2014, Culverwell et al., 2011, Truong et al., 2005, Bhargava et al., 2011, Blodgett et al., 2011, and Fukui et al., 2005) including relatively high false negative rates depending on tumor size and variable levels of FDG uptake in tumors and normal tissues. High physiologic uptake of FDG typically occurs in the brain, heart, kidneys, and urinary tract, obscuring the tumor signal from areas adjacent to these tissues (Truong et al., 2014). In head and neck cancer, high FDG uptake in Waldeyer's ring (nasopharyngeal, palatine and lingual tonsils), salivary glands, striated muscle, brown fat, or inflammation/infection all contribute to false positive signals (Cohade et al., 2003 and Perkins et al., 2013). For tumors less than 1 cm, inadequate accumulation of FDG in tumors over the surrounding normal tissues often leads to false negatives (Cook et al., 2004, Purohit et al., 2014, Blodgett et al., 2011, and Gould et al., 2001). In addition, skull base tumors in the vicinity of highly metabolic brain parenchyma or oropharyngeal and nasopharyngeal cancers in FDG-avid tonsillar tissue may yield false negative diagnoses (Harvey et al., 2010, Schoder, 2013, Castaigne et al., 2006, and Schmalfuss, 2012). The variability of FDG uptake, overlap in retention, and temporal fluctuations in metabolism for both normal and tumor tissues significantly limits the accuracy of FDG PET in cancer detection.

Previously reported was an indocyanine green (ICG)-encoded ultra pH sensitive (UPS) nanoprobe for the broad detection of a wide range of solid cancers by near infrared fluorescence imaging (Zhao et al., 2016). This optical tracer exploits the phase transition of the polymers to quench and unquench the fluorescence of dyes conjugated to the hydrophobic portion of the polymers. This optical output is discrete, all on or off with no intermediate values, leading to the high specificity and sensitivity in tumor detection. However, it was unclear whether the phase transition behavior of the polymers could be harnessed to generate a response or output other than fluorescence.

Due to the unmet clinical need for the development of broad and cancer-specific strategies for cancer detection across different tumor types with improved sensitivity and specificity, new polymers that can generate pH responsive systems for the imaging of tumors are of value to the development of diagnostic and imaging protocols.

### SUMMARY

In some aspects, the present disclosure provides polymers of the formula:

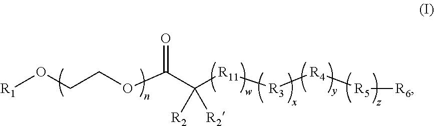

wherein:
R₁ is hydrogen, alkyl_{(C≤12)}, cycloalkyl_{(C≤12)}, substituted alkyl_{(C≤12)}, substituted cycloalkyl_{(C≤12)}, or

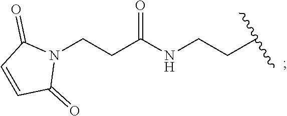 [SMILES: *CCNC(=O)CCN1C(=O)C=CC1=O]

n is an integer from 1 to 500;
R₂ and R₂′ are each independently selected from hydrogen, alkyl_{(C≤12)}, cycloalkyl_{(C≤12)}, substituted alkyl_{(C≤12)}, or substituted cycloalkyl_{(C≤12)};
R₃ and R₁₁ are each independently a group of the formula:

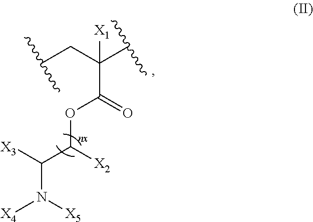

wherein:
   nₓ is 1-10;
   X₁, X₂, and X₃ are each independently selected from hydrogen, alkyl_{(C≤12)}, cycloalkyl_{(C≤12)}, substituted alkyl_{(C≤12)}, or substituted cycloalkyl_{(C≤12)}; and
   X₄ and X₅ are each independently selected from alkyl_{(C≤12)}, cycloalkyl_{(C≤12)}, aryl_{(C≤12)}, heteroaryl_{(C≤12)} or a substituted version of any of these groups, or X₄ and X₅ are taken together and are alkanediyl_{(C≤12)}, alkoxydiyl_{(C≤12)}, alkylaminodiyl_{(C≤12)}, or a substituted version of any of these groups;
w is an integer from 0 to 150;
x is an integer from 1 to 150;
R₄ is a group of the formula:

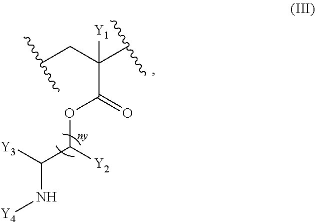

wherein:
   n_{y} is 1-10;
   Y₁, Y₂, and Y₃ are each independently selected from hydrogen, alkyl_{(C≤12)}, cycloalkyl_{(C≤12)}, substituted alkyl_{(C≤12)}, or substituted cycloalkyl_{(C≤12)}; and Y₄ is a dye or a fluorescence quencher;
y is an integer from 1-6;
R₅ is a group of the formula:

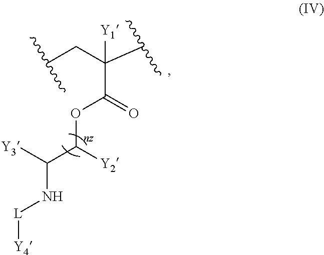

wherein:
   n_{z} is 1-10;
   Y₁′, Y₂′, and Y₃′ are each independently selected from hydrogen, alkyl_{(C≤12)}, cycloalkyl_{(C≤12)}, substituted alkyl_{(C≤12)}, or substituted cycloalkyl_{(C≤12)}; and
   Y₄′ is a metal chelating group;
   L is a covalent bond; or
      alkanediyl_{(C≤12)}, arenediyl_{(C≤12)}, -alkanediyl_{(C≤12)}-arenediyl_{(C≤12)}—NC(S)—, -alkanediyl_{(C≤12)}-arenediyl_{(C≤12)}-C(O)—, or a substituted version of any of these groups;
z is an integer from 1-6; and
R₆ is hydrogen, halo, hydroxy, alkyl_{(C≤12)}, or substituted alkyl_{(C≤12)},
   wherein R₁₁, R₃, R₄, and R₅ can occur in any order within the polymer.

In some embodiments, the polymer is defined by the formula wherein:
R₁ is hydrogen, alkyl_{(C≤12)}, substituted alkyl_{(C≤12)}, or

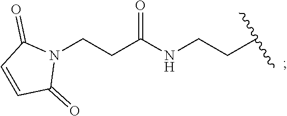 [SMILES: *CCNC(=O)CCN1C(=O)C=CC1=O]

n is an integer from 10 to 500;
R₂ and R₂′ are each independently selected from hydrogen, alkyl_{(C≤12)}, or substituted alkyl_{(C≤12)};
R₃ and R₁₁ are each independently a group of the formula:

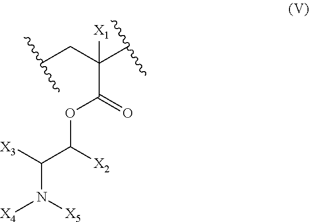

wherein:
   X₁, X₂, and X₃ are each independently selected from hydrogen, alkyl_{(C≤12)}, or substituted alkyl_{(C≤12)}; and
   X₄ and X₅ are each independently selected from alkyl_{(C≤12)}, aryl_{(C≤12)}, heteroaryl_{(C≤12)} or a substituted version of any of these groups, or X₄ and X₅ are taken together and are alkanediyl_{(C≤8)}, alkoxydiyl_{(C≤8)}, alkylaminodiyl_{(C≤8)}, or a substituted version of any of these groups;
x is an integer from 1 to 100;
w is an integer from 0 to 100;
R₄ is a group of the formula:

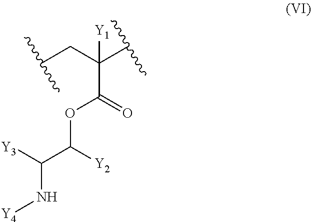

wherein:
   Y₁, Y₂, and Y₃ are each independently selected from hydrogen, alkyl_{(C≤12)}, or substituted alkyl_{(C≤12)}; and
   Y₄ is a dye or a fluorescence quencher;
y is an integer from 1 to 6;
R₅ is a group of the formula:

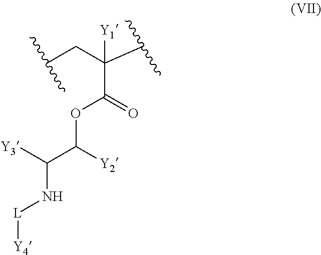

wherein:
   Y₁′, Y₂′, and Y₃′ are each independently selected from hydrogen, alkyl_{(C≤12)}, substituted alkyl_{(C≤12)}; and
   Y₄′ is a metal chelating group;
   L is a covalent bond; or
      alkanediyl_{(C≤12)}, arenediyl_{(C≤12)}, -alkanediyl_{(C≤12)}-arenediyl_{(C≤12)}—NC(S)—, -alkanediyl_{(C≤12)}-arenediyl_{(C≤12)}-C(O)—, or a substituted version of any of these groups;
z is an integer from 1-6; and
R₆ is hydrogen, halo, alkyl_{(C≤12)}, or substituted alkyl_{(C≤12)}, wherein R₁₁, R₃, R₄, and R₅ can occur in any order within the polymer.

In some embodiments, the polymer is further defined by the formula wherein:
R₁ is hydrogen, alkyl_{(C≤8)}, substituted alkyl_{(C≤8)}, or

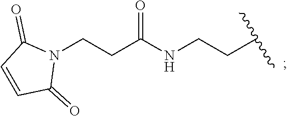 [SMILES: *CCNC(=O)CCN1C(=O)C=CC1=O]

n is an integer from 10 to 200;
R₂ and R₂′ are each independently selected from hydrogen, alkyl_{(C≤8)}, or substituted alkyl_{(C≤8)};
R₃ and R₁₁ are each independently a group of the formula:

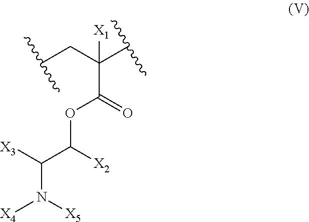

wherein:
   X₁, X₂, and X₃ are each independently selected from hydrogen, alkyl_{(C≤8)}, or substituted alkyl_{(C≤8)}; and
   X₄ and X₅ are each independently selected from alkyl_{(C≤12)}, aryl_{(C≤12)}, heteroaryl_{(C≤12)} or a substituted version of any of these groups, or X₄ and X₅ are taken together and are alkanediyl_{(C≤8)} or substituted alkanediyl_{(C≤8)};
x is an integer from 1 to 100;
w is an integer from 0 to 100;
R₄ is a group of the formula:

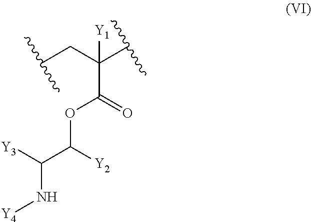

wherein:
   Y₁, Y₂, and Y₃ are each independently selected from hydrogen, alkyl_{(C≤8)}, or substituted alkyl_{(C≤8)}; and
   Y₄ is a dye or a fluorescence quencher;
y is an integer from 1 to 6;
R₅ is a group of the formula:

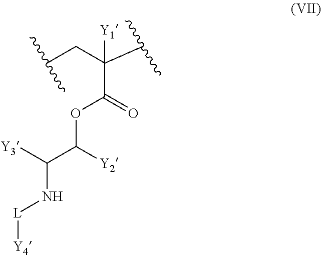

wherein:
   Y₁′, Y₂′, and Y₃′ are each independently selected from hydrogen, alkyl_{(C≤8)}, substituted alkyl_{(C≤8)}; and
   Y₄′ is a metal chelating group;
   L is a covalent bond; or
      alkanediyl_{(C≤12)}, arenediyl_{(C≤12)}, -alkanediyl_{(C≤12)}-arenediyl_{(C≤12)}—NC(S)—, -alkanediyl_{(C≤12)}-arenediyl_{(C≤12)}-C(O)—, or a substituted version of any of these groups;
z is an integer from 1-6; and
R₆ is hydrogen, halo, alkyl_{(C≤6)}, or substituted alkyl_{(C≤6)}, wherein R₁₁, R₃, R₄, and R₅ can occur in any order within the polymer.

In some embodiments, R₁ is hydrogen. In other embodiments, R₁ is alkyl_{(C≤6)}, such as methyl. In still other embodiments, R₁ is

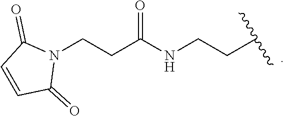 [SMILES: *CCNC(=O)CCN1C(=O)C=CC1=O]

In some embodiments, R₂ is alkyl_{(C≤6)}, such as methyl. In some embodiments, R₂′ is alkyl_{(C≤6)}, such as methyl. In some embodiments, R₃ or R₁₁ is further defined by the formula:

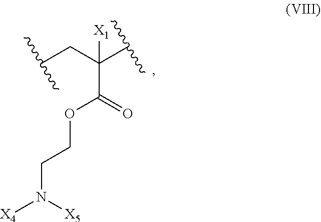

wherein:
X₁ is selected from hydrogen, alkyl_{(C≤8)}, or substituted alkyl_{(C≤8)}; and
X₄ and X₅ are each independently selected from alkyl_{(C≤12)}, aryl_{(C≤12)}, heteroaryl_{(C≤12)} or a substituted version of any of these groups, or X₄ and X₅ are taken together and are alkanediyl_{(C≤8)} or substituted alkanediyl_{(C≤8)}.

In some embodiments, X₁ is alkyl_{(C≤6)}, such as methyl. In some embodiments, X₄ is alkyl_{(C≤8)}, such as methyl, ethyl, propyl, butyl, or pentyl. In some embodiments, X₄ is n-propyl. In other embodiments, X₄ is isopropyl. In other embodiments, X₄ is ethyl. In some embodiments, X₅ is alkyl_{(C≤8)}, such as methyl, ethyl, propyl, butyl, or pentyl. In some embodiments, X₅ is n-propyl. In other embodiments, X₅ is isopropyl. In other embodiments, X₅ is ethyl. In some embodiments, R₃ and R₁₁ are not same group.

In some embodiments, R₄ is further defined by the formula:

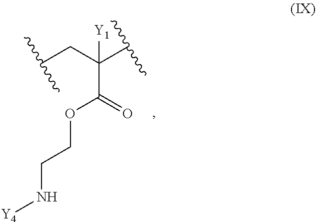

wherein: Y₁ is selected from hydrogen, alkyl_{(C≤8)}, or substituted alkyl_{(C≤8)}; and Y₄ is a dye or a fluorescence quencher. In some embodiments, Y₁ is alkyl_{(C≤6)}, such as methyl. In some embodiments, Y₄ is a dye. In some embodiments, Y₄ is fluorescent dye. In some embodiments, the fluorescent dye is a coumarin, fluorescein, rhodamine, xanthene, BODIPY®, Alexa Fluor®, or cyanine dye. In some embodiments, the fluorescent dye is indocyanine green, AMCA-x, Marina Blue, PyMPO, Rhodamine Green™, Tetramethylrhodamine, 5-carboxy-X-rhodamine, Bodipy493, Bodipy TMR-x, Bodipy630, Cyanine3.5, Cyanine5, Cyanine5.5, or Cyanine7.5. In some embodiments, the fluorescent dye is indocyanine green.

In some embodiments, Y₄ is a fluorescence quencher, such as QSY7, QSY21, QSY35, BHQ1, BHQ2, BHQ3, TQ1, TQ2, TQ3, TQ4, TQ5, TQ6, or TQ7. In some embodiments, each R₁₁ is incorporated consecutively to form a block. In some embodiments, each R₃ is incorporated consecutively to form a block. In some embodiments, each R₁₁ is present as a block and each R₃ is present as a block. In other embodiments, each R₁₁ and each R₃ are randomly incorporated within the polymer.

In some embodiments, R₅ is further defined by the formula:

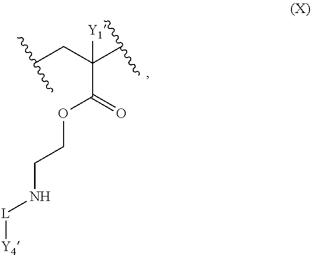

wherein:
Y₁′ is selected from hydrogen, alkyl_{(C≤8)}, substituted alkyl_{(C≤8)};
Y₄′ is a metal chelating group; and
L is a covalent bond; or alkanediyl_{(C≤12)}, arenediyl_{(C≤12)}, -alkanediyl_{(C≤12)}-arenediyl_{(C≤12)}—NC(S)—, -alkanediyl_{(C≤12)}-arenediyl_{(C≤12)}-C(O)—, or a substituted version of any of these groups.

In some embodiments, Y₁′ is alkyl_{(C≤6)}, such as methyl. In some embodiments, L is a covalent bond. In other embodiments, L is alkanediyl_{(C≤12)}, substituted alkanediyl_{(C≤12)}, arenediyl_{(C≤12)}, or substituted arenediyl_{(C≤12)}. In still other embodiments, L is -alkanediyl_{(C≤12)}-arenediyl_{(C≤12)}—NC(S)—. In further embodiments, L is -alkanediyl_{(C≤12)}-benzenediyl-NC(S)—, such as —CH₂-1,4-benzenediyl-NC(S)—. In some embodiments, Y₄′ is DOTA, TETA, Diamsar, NOTA, NETA, TACN-TM, DTPA, TRAP, NOPO, AAZTA, DATA, HBED, SHBED, BPCA, CP256, DFO, PCTA, HEHA, PEPA, or a derivative thereof. In some embodiments, Y₄′ is a metal chelating group wherein the metal chelating group is a nitrogen containing macrocycle. In further embodiments, the nitrogen containing macrocycle is a compound of the formula:

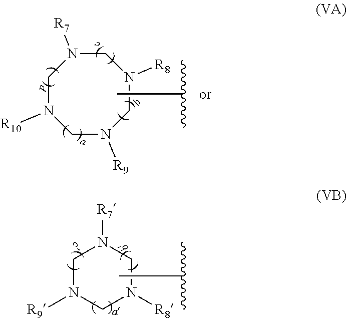

wherein:
R₇, R₈, R₉, R₁₀, R₇′, R₈′, and R₉′ are each independently selected from hydrogen, alkyl_{(C≤12)}, acyl_{(C≤12)}, -alkanediyl_{(C≤12)}-acyl_{(C≤12)}, or a substituted version of any of these groups; or
R₇ is taken together with one of R₈, R₉, or R₁₀ and is alkanediyl_{(C≤6)}; or
R₈ is taken together with one of R₇, R₉, or R₁₀ and is alkanediyl_{(C≤6)}; or
R₉ is taken together with one of R₇, R₈, or R₁₀ and is alkanediyl_{(C≤6)}; or
R₁₀ is taken together with one of R₇, R₈, or R₉ and is alkanediyl_{(C≤6)}; or
R₇′ is taken together with one of R₈′ or R₉′ and is alkanediyl_{(C≤6)}; or
R₈′ is taken together with one of R₇′ or R₉′ and is alkanediyl_{(C≤6)}; or
R₉′ is taken together with one of R₇′ or R₈′ and is alkanediyl_{(C≤6)}; and
a, b, c, d, a′, b′, and c′ are each independently selected from 1, 2, 3, or 4.

In some embodiments, a, b, c, d, a′, b′, and c′ are each independently selected from 2 or 3.

In some embodiments, the metal chelating group is:

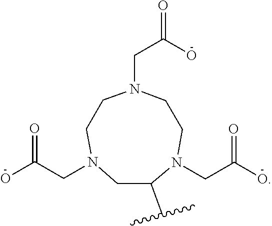

In some embodiments, the metal chelating group is bound to a metal ion to form a metal complex. In some embodiments, the metal ion is a radionuclide or radiometal. In some embodiments, the metal ion is suitable for PET or SPECT imaging. In some embodiments, the metal ion is a transition metal ion. In some embodiments, the metal ion is a copper ion, a gallium ion, a scandium ion, an indium ion, a lutetium ion, a ytterbium ion, a zirconium ion, a bismuth ion, a lead ion, a actinium ion, or a technetium ion. In some embodiments, the metal ion is an isotope selected from ^{99m}Tc, ⁶⁰Cu, ⁶¹Cu, ⁶²Cu, ⁶⁴Cu, ⁸⁶Y, ⁹⁰Y, ⁸⁹Zr, ⁴⁴Sc, ⁴⁷Sc, ⁶⁶Ga, ⁶⁷Ga, ⁶⁸Ga, ¹¹¹In, ¹⁷⁷Lu, ²²⁵Ac, ²¹²Pb, ²¹²Bi, ²¹³Bi, ¹¹¹In, ^{114m}In, ¹¹⁴In, ¹⁸⁶Re, or ¹⁸⁸Re. In some embodiments, the transition metal ion is a copper(II) ion. In some embodiments, the copper(II) ion is a ⁶⁴Cu²⁺ ion. In some embodiments, the metal complex is:

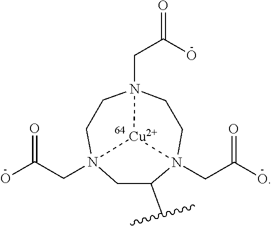

In some embodiments, n is 75-150. In further embodiments, n is 100-125. In some embodiments, x is 1-99. In further embodiments, x is from 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-199 or any range derivable therein. In some embodiments, y is 1, 2, 3, 4, or 5. In some embodiments, y is 1 or 2. In some embodiments, y is 1. In some embodiments, z is 1, 2, 3, 4, or 5. In some embodiments, z is 1 or 2. In some embodiments, z is 2. In some embodiments, each R₁₁, R₃, R₄, and R₅ can occur in any order within the polymer. In other embodiments, each R₁₁, R₃, R₄, and R₅ occur in the order described in formula I. In some embodiments, w is 0.

In some embodiments, the polymer further comprises a targeting moiety. In further embodiments, targeting moiety is a small molecule, an antibody, an antibody fragment, or a signaling peptide. In some embodiments, R₃ and R₁₁ are selected from:

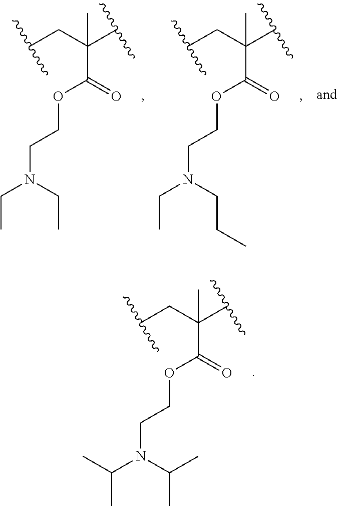 [SMILES: *CC(*)(C)C(=O)OCCN(CC)CC ; *CC(*)(C)C(=O)OCCN(CC)CCC ; *CC(*)(C)C(=O)OCCN(C(C)C)C(C)C]

In some embodiments, R₃ is:

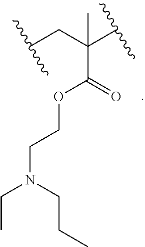 [SMILES: *CC(*)(C)C(=O)OCCN(CC)CCC]

In some embodiments, the polymer has a pH transition of 6.9. In some embodiments, the polymer is UPS_{6.9}.

In another aspect, the present disclosure provides micelles of a polymer as disclosed herein.

In another aspect, the present disclosure provides pH responsive systems comprising a micelle of a first polymer wherein the first polymer is a polymer of the present disclosure, wherein Y₄ is a dye, and wherein the micelle has a pH transition point and an emission spectrum. In some embodiments, the micelle further comprises a second polymer is a polymer as disclosed herein, wherein Y₄ is a fluorescence quencher. In some embodiments, the second polymer has the same formula as the first polymer except that Y₄ is a fluorescence quencher. In some embodiments, the micelle comprises a composition comprising a second polymer of the present disclosure, wherein the second polymer has a different formula than the first polymer. In some embodiments, Y₄ on the second polymer is a different dye than the Y₄ on the first polymer. In some embodiments, the micelle further comprises from 1 to 6 additional polymers provided that each polymer is unique that each polymer is different than the first polymer and the second polymer. In some embodiments, the pH transition point is between 3-9. In further embodiments, the pH transition point is between 4-8, such as 6.9. In some embodiments, the emission spectra is between 400-850 nm. In some embodiments, the system has a pH response (ΔpH_{10-90%}) of less than 1 pH unit. In further embodiments, the pH response is less than 0.25 pH units. In still further embodiments, the pH response is less than 0.15 pH units. In some embodiments, the fluorescence signal has a fluorescence activation ratio of greater than 25. In further embodiments, the fluorescence activation ratio is greater than 50.

In another aspect, the present disclosure provides methods of imaging the pH of an intracellular or extracellular environment comprising:
(a) contacting a pH responsive system of the present disclosure with the environment; and
(b) detecting one or more signals from the environment, wherein the detection of the signal indicates that the micelle has reached its pH transition point and disassociated.

In some embodiments, at least one of the one of more signals is positron emission. In some embodiments, at least one of the one of more signals is an optical signal, such as a fluorescent signal. In some embodiments, when the intracellular environment is imaged, the cell is contacted with the pH responsive system under conditions suitable to cause uptake of the pH responsive system. In some embodiments, the intracellular environment is part of a cell. In further embodiments, the part of the cell is lysosome or an endosome. In some embodiments, the extracellular environment is of a tumor or vascular cell. In some embodiments, the extracellular environment is intravascular or extravascular. In some embodiments, imaging the pH of the tumor environment comprises imaging the cancer-involved or sentinel lymph node or nodes. In further embodiments, imaging the cancer-involved or sentinel lymph node or nodes allows for the surgical resection of the tumor and staging of the tumor metastasis. In some embodiments, imaging the pH of the tumor environment allows determination of the tumor size and margins. In further embodiments, imaging the pH of the tumor environment allows for more precise removal of the tumor during surgery.

In some embodiments, the method further comprises:
(a) contacting the cell with a compound of interest;
(b) detecting one or more signals in the environment; and
(c) determining whether a change in the one or more signals occurred following contacting the cell with the compound of interest.

In some embodiments, at least one of the one or more signals is an optical signal. In some embodiments, at least one of the one or more signals is positron emission. In some embodiments, the compound of interest is a drug, antibody, peptide, protein, nucleic acid, or small molecule.

In still another aspect, the present disclosure provides methods of delivering a compound of interest to a target cell comprising:
(a) encapsulating the compound of interest with a pH responsive system of a polymer of the present disclosure; and
(b) contacting the target cell with the pH responsive system under such conditions that the pH of the target cell triggers the disassociation of the pH responsive system and release of the compound, thereby delivering the compound of interest.

In some embodiments, the compound of interest is delivered into the cell. In other embodiments, the compound of interest is delivered to the cell. In some embodiments, the compound of interest is a drug, antibody, peptide, protein, nucleic acid, or small molecule. In some embodiments, the method further comprises administering the pH responsive system to a patient.

In yet another aspect, the present disclosure provides methods of resecting a tumor in a patient comprising:
(a) administering to the patient an effective dose of a pH responsive system of the present disclosure;
(b) detecting one or more signals for the patient; wherein the one of more signals indicate the presence of a tumor; and
(c) resecting the tumor via surgery.

In some embodiments, at least one of the one or more signals is an optical signal. In some embodiments, at least one of the one or more signals is positron emission. In some embodiments, the one or more signals indicate the margins of the tumor. In some embodiments, the tumor is 90% resected. In further embodiments, the tumor is 95% resected. In still further embodiments, the tumor is 99% resected. In some embodiments, the tumor is a solid tumor. In some embodiments, the solid tumor is from a cancer. In some embodiments, the cancer is a breast cancer, a head and neck cancer, or a brain cancer. In some embodiments, the cancer is head and neck squamous cell carcinoma. In some embodiments, the pH responsive system is comprised of a polymer of the formula:

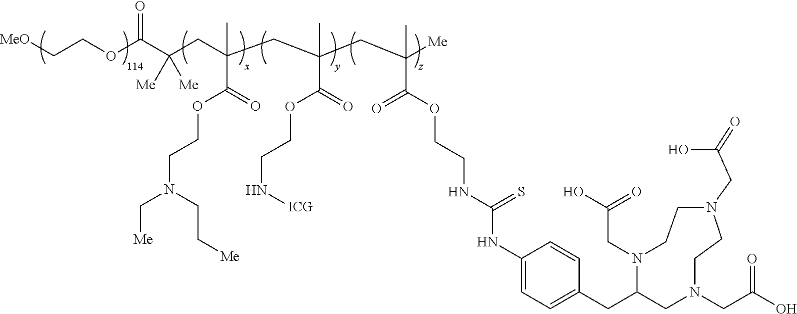

wherein: x is an integer from 30 to 150, y is 1 or 2, z is 1 or 2; x, y, and z are randomly distributed throughout the polymer; ICG is the fluorescent dye indocyanine green.

In yet another aspect, the present disclosure provides methods of treating a cancer susceptible to endosomal/lysosomal pH arrest in a patient comprising administering to the patient in need thereof a pH responsive system of the present disclosure. In some embodiments, the cancer is a lung cancer, such as a non-small cell lung cancer. In some embodiments, the method is sufficient to induce apoptosis.

In still another aspect, the present disclosure provides methods of identifying the tumor acidosis pathway comprising:
(a) contacting a pH responsive system comprising one or more micelles of the present disclosure with a cell or a cellular environment;
(b) contacting the cell with an inhibitor of the pH regulatory pathway;
(c) detecting a signal from the cell or cellular environment, wherein the detection of the signal indicates that one of the micelles has reached its pH transition point and disassociated; and
(d) correlating the signal with a modification in the tumor acidosis pathway.

In some embodiments, the signal is an optical signal, such as fluorescence. In some embodiments, the signal is positron emission. In some embodiments, the inhibitor of the pH regulatory pathway is an inhibitor of a monocarboxylate transporter, a carbonic anhydrase, an anion exchanger, a Na⁺-bicarbonate exchanger, a Na⁺/H⁺ exchanger, or a V-ATPase. In some embodiments, the one or more micelles comprise a polymer with two or more fluorophores attached to the polymer backbone. In further embodiments, the method comprises one micelle and the micelle comprises two or more polymers with different fluorophores or different R₃ groups. In some embodiments, the micelle comprises two or more polymers with different fluorophores and different R₃ groups.

In still another aspect, the present disclosure provides methods of imaging a patient to determine the presence of a tumor comprising:
(a) contacting a pH responsive system comprising one or more micelles of the present disclosure with the tumor;
(b) collecting one or more PET or SPECT imaging scans; and
(c) collecting one or more optical imaging scans, wherein the detection of the optical signal indicates that one of the micelles has reached its pH transition point and disassociated;
   wherein the one or more PET or SPECT imaging scans and the one or more optical imaging scans result in the identification of a tumor. In some embodiments, the optical imaging scans are collected before the PET or SPECT imaging scans. In other embodiments, the optical imaging scans are collected after the PET or SPECT imaging scans. In still other embodiments, the optical imaging scans are collected simultaneously with the PET or SPECT imaging scans. In some embodiments, the imaging scans are PET imaging scans. In other embodiments, the imaging scans are SPECT imaging scans. In some embodiments, the metal chelating group is bound to a ⁶⁴Cu ion. In some embodiments, the metal chelating group is a nitrogen containing macrocycle. In some embodiments, the nitrogen containing macrocycle is:

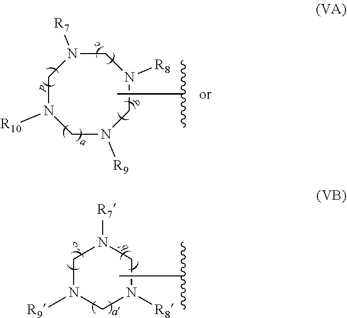

wherein: R₇, R₈, R₉, R₁₀, R₇′, R₈′, R₉′ a, b, c, d, a′, b′, and c′ are as defined above. In some embodiments, the nitrogen containing macrocycle is:

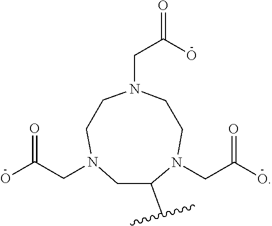

In some aspects, the present disclosure provides methods of determining the efficacy of a cancer treatment therapy comprising:
(a) administering a pH responsive system comprising one or more micelles of the present disclosure to a patient, wherein the patient has a tumor;
(b) collecting one or more PET or SPECT imaging scans;
(c) collecting one or more optical imaging scans, wherein the detection of the optical signal indicates that one of the micelles has reached its pH transition point and disassociated;
(d) administering the cancer treatment therapy;
(e) repeating steps (a)-(c) to determine the efficacy of the cancer treatment therapy.

In some embodiments, the cancer treatment therapy is chemotherapy or radiation therapy.

In some embodiments, chemotherapy comprises administration of a chemotherapeutic agent that modulates the tumor acidosis pathway.

In yet another aspect, the present disclosure provides methods of treating or preventing a disease or disorder in a patient in need thereof comprising administering to the patient a polymer, micelle, or pH responsive system described herein. In some embodiments, the polymer, micelle, or pH responsive system comprises a radionuclide, such as ⁹⁰Y or ¹⁷⁷Lu. In some embodiments, the polymer, micelle, or pH responsive system further comprises a second therapeutic agent.

As used herein, “pH responsive system,” “micelle,” “pH-responsive micelle,” “pH-sensitive micelle,” “pH-activatable micelle” and “pH-activatable micellar (pHAM) nanoparticle” are used interchangeably herein to indicate a micelle comprising one or more block copolymers, which disassociates depending on the pH (e.g., above or below a certain pH). As a non-limiting example, at a certain pH, the block copolymer is substantially in micellar form. As the pH changes (e.g., decreases), the micelles begin to disassociate, and as the pH further changes (e.g., further decreases), the block copolymer is present substantially in disassociated (non-micellar) form.

As used herein, “pH transition range” indicates the pH range over which the micelles disassociate.

As used herein, “pH transition value” (pHₜ) indicates the pH at which half of the micelles are disassociated.

It is contemplated that any method or composition described herein can be implemented with respect to any other method or composition described herein.

The terms “comprise” (and any form of comprise, such as “comprises” and “comprising”), “have” (and any form of have, such as “has” and “having”), “contain” (and any form of contain, such as “contains” and “containing”), and “include” (and any form of include, such as “includes” and “including”) are open-ended linking verbs. As a result, a method, composition, kit, or system that “comprises,” “has,” “contains,” or “includes” one or more recited steps or elements possesses those recited steps or elements but is not limited to possessing only those steps or elements; it may possess (i.e., cover) elements or steps that are not recited. Likewise, an element of a method, composition, kit, or system that “comprises,” “has,” “contains,” or “includes” one or more recited features possesses those features but is not limited to possessing only those features; it may possess features that are not recited.

Any embodiment of any of the present methods, composition, kit, and systems may consist of or consist essentially of—rather than comprise/include/contain/have—the described steps and/or features. Thus, in any of the claims, the term “consisting of” or “consisting essentially of” may be substituted for any of the open-ended linking verbs recited above, in order to change the scope of a given claim from what it would otherwise be using the open-ended linking verb.

The polymers of the present disclosure may be depicted in various protonation states. As would be recognized by a skilled artisan, molecules exist in different protonation states at different pH values. The depiction of a molecule in one protonation state does not mean that that molecule solely exists in that protonation state at that pH value or another pH value. Thus, the present polymers are contemplated to encompass all feasible protonation states. For example, an amine may be represented as either protonated or unprotonated or a carboxylic acid group may be depicted as either as the free acid or a carboxylate.

The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the standard deviation of error for the device or method being employed to determine the value. Following long-standing patent law, the words “a” and “an,” when used in conjunction with the word “comprising” in the claims or specification, denotes one or more, unless specifically noted.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1C show the synthesis and characterization of UPS_{6.9} nanoprobes. FIG. 1A shows schematic syntheses of NOTA- and ICG-conjugated PEG-b-PEPA block copolymers. FIG. 1B. shows radio-TLC chromatogram of UPS_{6.9} nanoprobes before and after centrifugation purification. Labeling efficiency was measured by instant thin layer chromatography (ITLC) with saline as the developing eluent and was shown to be more than 95%. FIG. 1C shows dynamic light scattering analysis of UPS_{6.9} nanoprobes at pH 7.4 and 6.5 (above and below the pH transition threshold, respectively).

FIGS. 2A & 2B show synthesis and characterization of ⁶⁴Cu-PEG-PLA nanoprobes. FIG. 2A shows schematic syntheses of NOTA-conjugated PEG-b-PLA block polymers FIG. 2B shows dynamic light scattering analysis of PEG-b-PLA nanoprobes for the measurement of size and size distribution in different pHs.

FIGS. 3A-3D show all-or-nothing proton distribution of UPS_{6.9} nanoprobes. FIG. 3A shows pH titration curve of UPS_{6.9} showed a reversible and sharp pH transition in saline solution. FIG. 3B shows reversible hydrodynamic size change of UPS_{6.9} along the pH titration coordinate. FIG. 3C shows quantification of proton binding cooperativity by the UPS copolymers yields a Hill coefficient of 38. FIG. 3D shows quantification of unimer and micelle charge states of UPS_{6.9} at a protonation degree of 50%. Protons were distributed divergently where unimers were highly charged (˜90%) and micelles were almost neutral.

FIGS. 4A-4E show irreversible capture of UPS nanoprobes by serum protein binding and cancer cell uptake after pH activation. FIG. 4A shows schematic illustration of acid-activated protein binding and membrane adhesion of UPS tracers leading to their sequestration inside lysosomes of cancer cells. FIG. 4B shows irreversible arrest of UPS tracers in the unimer state in the presence of serum proteins even after pH reversal to 7.4, compared to the reversible fluorescence changes in the absence of serum proteins. FIG. 4C shows autoradiography images of HN5 cells incubated with ⁶⁴Cu-UPS_{6.9} and ⁶⁴Cu-PEG-PLA nanoparticles (both at 25 μg/mL) at pH 6.5 and 7.4 over time. FIG. 4D shows significantly higher cell uptake of UPS_{6.9} was found at pH 6.5 than 7.4, as well as ⁶⁴Cu-PEG-PLA tracers at either pH. Data are presented as mean±s.d (n=3); **P<0.01 compared to other groups. FIG. 4E shows confocal microscopy showed UPS_{6.9} were mostly bound to cell membranes at 5 mins, followed by lysosome colocalization at 60 mins after incubation with HN5 cells. Scale bar=50 μm.

FIG. 5 shows autoradiography images of HN5 cells incubated with ⁶⁴Cu-UPS_{6.9} and ⁶⁴Cu-PEG-PLA nanoparticles (both at 25 μg/mL) at pH 6.5 and 7.4 over time.

FIG. 6 shows spatio-temporal characterization of ⁶⁴Cu-UPS_{6.9} accumulation in HN5 tumors. ⁶⁴Cu-UPS_{6.9} nanosensor (0.1 mCi) was injected through the tail vein. At 30 mins, 3 h, and 24 h, HN5 tumors were removed and tissue distribution of ⁶⁴Cu-UPS_{6.9} was analyzed by autoradiography. H&E histology slides were also provided for tumor demarcation. Scare bars are 2.5 mm.

FIGS. 7A-7C show the “capture and integration” strategy allowed binary detection of a brain tumor at both macroscopic (animal) and microscopic (subcellular) levels. FIG. 7A shows PET imaging of an orthotopic 73C murine brain tumor in a C57BL/6 mouse by ⁶⁴Cu-UPS_{6.9}. FIG. 7B shows correlation of H&E, GFP fluorescence, autoradiography (AR) and ICG fluorescence imaging of brain tumor slide supports the cancer-specific imaging by UPS nanoprobes. Scale bar is 2.5 mm in H&E image and applies to all the images in FIG. 7B. FIG. 7C shows fluorescence microscopy analysis of UPS tracer in brain tumors. Co-localization of ICG with GPF signals shows the effective crossing of blood tumor barrier and uptake of UPS in GFP-labeled 73C brain cancer cells. Scale bar=50 μm.

FIG. 8 shows PET imaging of GFP-transfected 73C glioblastoma orthotopic tumor models by ⁶⁴Cu-UPS_{6.9}, followed by fluorescence imaging of brain slides, correlated with histology. Scale bar is 2.5 mm.

FIGS. 9A-9C show non-invasive digitization of tumor acidotic signals by PET. FIG. 9A shows cancer-specific detection of various small tumor nodules (10-20 mm³) by i.v. administered ⁶⁴Cu-UPS tracers. Orthotopic HN5 and FdDu head and neck cancer and 4T1 triple negative breast cancer were clearly visualized. Liver and spleen are the other major organs for UPS uptake. FDG-PET image showed high false rates in the head and neck region (BR, brain; BF, brown fat). FIG. 9B shows PET quantification of CNR ratio for ⁶⁴Cu-UPS_{6.9} on different tumor models. FIG. 9C shows PET quantification of CNR ratio for HN5 bearing mice administered with ⁶⁴Cu-UPS_{6.9}, FDG and ⁶⁴Cu-PEG-PLA respectively. Data are presented as individual data points plus mean±s.d (n=3); **P<0.01 compared to other groups.

FIGS. 10A-10C show detection of HN5 orthotopic tumors with great PET contrast 24 hours after administration of ⁶⁴Cu-UPS_{6.9} nanoprobes. FIG. 10A shows PET/CT imaging of HN5 orthotopic tumor models. FIG. 10B shows correlation of H&E and autoradiography imaging of HN5 tumor slides showed the cancer-specificity by ⁶⁴Cu-UPS_{6.9} nanoprobes. FIG. 10C shows biodistribution profiles quantified by PET signals in different organs (n=3) of ⁶⁴Cu-UPS_{6.9} 24 hours after intravenous injection. Scale bar is 2.5 mm and 500 m in H&E and enlarged images, respectively.

FIGS. 11A-11C show detection of FaDu orthotopic tumors with great PET contrast 24 hours after administration of ⁶⁴Cu-UPS_{6.9} nanoprobes. FIG. 11A shows PET/CT imaging of FaDu orthotopic tumor models. FIG. 11B shows correlation of H&E and autoradiography imaging of FaDu tumor slides showed the cancer-specificity by ⁶⁴Cu-UPS_{6.9} nanoprobes. FIG. 11C shows biodistribution profiles quantified by PET signals in different organs (n=3) of ⁶⁴Cu-UPS_{6.9} 24 hours after intravenous injection. Scale bar is 2.5 mm and 500 m in H&E and enlarged images, respectively.

FIGS. 12A-12C shows detection of 4T1 orthotopic tumors with great PET contrast 24 hours after administration of ⁶⁴Cu-UPS_{6.9} nanoprobes. FIG. 12A shows PET/CT imaging of 4T1 orthotopic tumor models. FIG. 12B shows correlation of H&E and autoradiography imaging of 4T1 tumor slides showed the cancer-specificity by ⁶⁴Cu-UPS_{6.9} nanoprobes. FIG. 12C shows biodistribution profiles quantified by PET signals in different organs (n=3) of ⁶⁴Cu-UPS_{6.9} 24 hours after intravenous injection. Scale bar is 2.5 mm and 250 m in H&E and enlarged images, respectively.

FIGS. 13A & 13B show FDG-PET/CT imaging of HN5 orthotopic tumor models. FIG. 13A shows PET/CT imaging of 4T1 orthotopic tumor models. FIG. 13B shows biodistribution profiles quantified by PET signals in different organs (n=3) of FDG 1 hour after intravenous injection.

FIGS. 14A-14C show detection of HN5 orthotopic tumors with less PET contrast 24 hours after administration of ⁶⁴Cu-PEG-b-PLA nanoprobes. FIG. 14A shows PET/CT imaging of HN5 orthotopic tumor models. FIG. 14B shows correlation of H&E and autoradiography imaging of HN5 tumor slides showed small tumor contrast by ⁶⁴Cu-PEG-b-PLA nanoprobes. FIG. 14C shows biodistribution profiles quantified by PET signals in different organs (n=3) of ⁶⁴Cu-PEG-b-PLA nanoprobes 24 hours after intravenous injection. Scale bar is 2.5 mm in H&E image.

FIG. 15 shows schematic for the capture and integration algorithm to convert perpetual spatio-temporal fluctuations of tumor acidotic signals into step functions of binary response (0 and 1) by the pH threshold tracer.

FIG. 16 shows PET/CT imaging of small orthotopic HN5 tumors (˜20 mm³) by ⁶⁴Cu-UPS and FDG. Images were taken 24 h after intravenous injection of the probes. Yellow arrows indicate the location of HN5 tumors. The SUV scale applies to both images.

FIGS. 17A-C shows ⁶⁴Cu-UPS probes with tunable pH transitions. FIG. 17A shows a synthetic scheme of PEG-b-(PR-ICG-NOTA) copolymers. FIG. 17B shows the pH response of the PS probes for different PR compositions. FIG. 17C shows transition pH as a function of molar percentage of DPA establishes a standard curve for rational design of ⁶⁴Cu-UPS with pre-determined pH transitions.

### DESCRIPTION OF ILLUSTRATIVE EMBODIMENTS

In some aspects, the present disclosure provides a polymer which can form a pH responsive nanoparticle which dissembles above a particular transition pH. In some embodiments, these polymers comprise a mixture of different monomers which allow specific tailoring of the desired pH transition point (ΔpH_{10-90%}) of less than 0.2 pH units as well as develop pH probes for a range of pH transition points from about a pH of 4 to about a pH of 8. The wide range of pH transition points allows for a wide range of application including but not limited to vesicular trafficking, imaging of the pHₑ of tumors, delivering drug compounds to specific tissues, improving the visualization of a tumor to improve the ability of a surgeon to resect the tumor tissue, or study the maturation or development of endosomes/lysosomes. In some aspects, the polymers of the present disclosure comprise a metal chelating group and a dye or fluorescence quencher. In some aspects, the metal chelating group is chelated to a radionuclide, such as a radionuclide that emits positrons. In some aspects, the present disclosure provides methods of using these polymers in a pH responsive system as described above. Additional methods of using the polymers and the resultant pH responsive systems of the present disclosure are described in WO 2013/152059 and WO 2015/188157, which are incorporated herein by reference. In some embodiments, the compounds of the present invention have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

### A. Chemical Definitions

When used in the context of a chemical group: “hydrogen” means —H; “hydroxy” means —OH; “carboxy” means —C(═O)OH (also written as —COOH or —CO₂H); “halo” means independently —F, —Cl, —Br or —I; “amino” means —NH₂; “nitro” means —NO₂; “cyano” means —CN; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means=S; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, for example, the formula

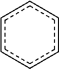

includes

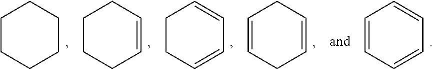

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

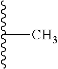

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

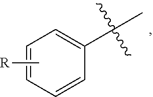

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

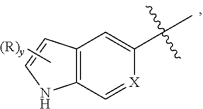

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and classes below, the following parenthetical subscripts further define the group/class as follows: “(Cn)” defines the exact number (n) of carbon atoms in the group/class. “(C≤n)” defines the maximum number (n) of carbon atoms that can be in the group/class, with the minimum number as small as possible for the group in question, e.g., it is understood that the minimum number of carbon atoms in the group “alkenyl_{(C≤8)}” or the class “alkene_{(C≤8)}” is two. For example, “alkoxy_{(C≤10)}” designates those alkoxy groups having from 1 to 10 carbon atoms. (Cn-n′) defines both the minimum (n) and maximum number (n′) of carbon atoms in the group. Similarly, “alkyl_{(C2-10)}” designates those alkyl groups having from 2 to 10 carbon atoms.

The term “saturated” as used herein means the compound or group so modified has no carbon-carbon double and no carbon-carbon triple bonds, except as noted below. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl).

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂—, are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the compound H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which one or more hydrogen atoms has been substituted with a halo group and no other atoms aside from carbon, hydrogen and halogen are present. The group, —CH₂C1 is a non-limiting example of a haloalkyl. The term “fluoroalkyl” is a subset of substituted alkyl, in which one or more hydrogen has been substituted with a fluoro group and no other atoms aside from carbon, hydrogen and fluorine are present. The groups, —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, a linear or branched cyclo or cyclic structure, no carbon-carbon double or triple bonds, and no atoms other than carbon and hydrogen. As used herein, the cycloalkyl group may contain one or more branching alkyl groups (carbon number limit permitting) attached to the ring system so long as the point of attachment is the ring system. Non-limiting examples of cycloalkyl groups include: —CH(CH₂)₂ (cyclopropyl), cyclobutyl, cyclopentyl, or cyclohexyl. The term “cycloalkanediyl” when used without the “substituted” modifier refers to a divalent saturated aliphatic group with one or two carbon atom as the point(s) of attachment, a linear or branched cyclo or cyclic structure, no carbon-carbon double or triple bonds, and no atoms other than carbon and hydrogen.

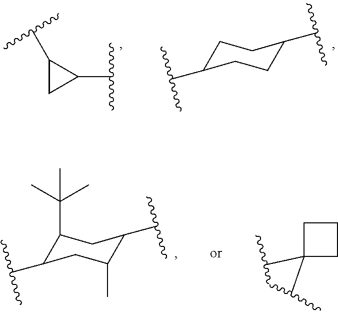

or are non-limiting examples of cycloalkanediyl groups. The term “cycloalkylidene” when used without the “substituted” modifier refers to the divalent group ═CRR′ in which R and R′ are taken together to form a cycloalkanediyl group with at least two carbons. Non-limiting examples of alkylidene groups include: ═C(CH₂)₂ and ═C(CH₂)₅. A “cycloalkane” refers to the compound H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The following groups are non-limiting examples of substituted cycloalkyl groups: —C(OH)(CH₂)₂,

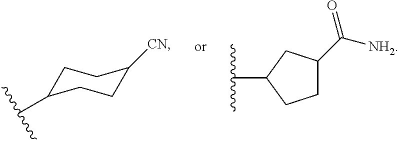 [SMILES: *C1CCC(C#N)CC1 ; *C1CCC(C(N)=O)C1]

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

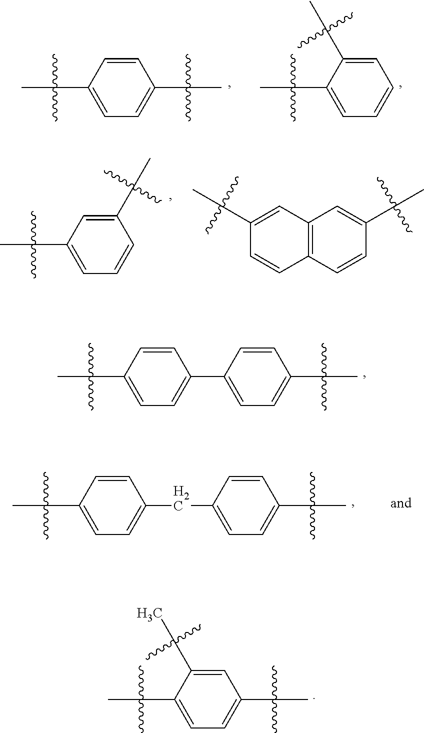

An “arene” refers to the compound H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl, pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

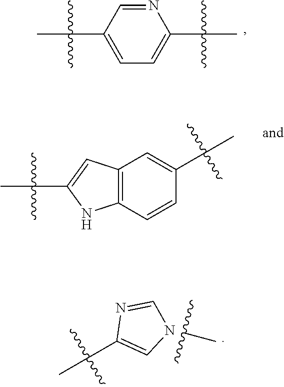

A “heteroarene” refers to the compound H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

The term “acyl” when used without the “substituted” modifier refers to the group —C(O)R, in which R is a hydrogen, alkyl, cycloalkyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples of alkoxy groups include: —OCH₃ (methoxy), —OCH₂CH₃ (ethoxy), —OCH₂CH₂CH₃, —OCH(CH₃)₂ (isopropoxy), and —OC(CH₃)₃ (tert-butoxy). The terms “cycloalkoxy”, “alkenyloxy”, “cycloalkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, cycloalkenyl, alkynyl, aryl, aralkyl, heteroaryl, heterocycloalkyl, and acyl, respectively. The term “alkoxydiyl” refers to the divalent group —O-alkanediyl-, —O-alkanediyl-O—, or -alkanediyl-O-alkanediyl-. The terms “alkylthio”, “cycloalkylthio”, and “acylthio” when used without the “substituted” modifier refers to the group —SR, in which R is an alkyl, cycloalkyl, and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy or cycloalkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

The term “alkylamino” when used without the “substituted” modifier refers to the group —NHR, in which R is an alkyl, as that term is defined above. Non-limiting examples of alkylamino groups include: —NHCH₃ and —NHCH₂CH₃. The term “dialkylamino” when used without the “substituted” modifier refers to the group —NRR′, in which R and R′ can each independently be the same or different alkyl groups, or R and R′ can be taken together to represent an alkanediyl. Non-limiting examples of dialkylamino groups include: —N(CH₃)₂, —N(CH₃)(CH₂CH₃), and N-pyrrolidinyl. The terms “alkoxyamino”, “cycloalkylamino”, “alkenylamino”, “cycloalkenylamino”, “alkynylamino”, “arylamino”, “aralkylamino”, “heteroarylamino”, “heterocycloalkylamino” and “alkylsulfonylamino” when used without the “substituted” modifier, refers to groups, defined as —NHR, in which R is alkoxy, cycloalkyl, alkenyl, cycloalkenyl, alkynyl, aryl, aralkyl, heteroaryl, heterocycloalkyl, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. The term “alkylaminodiyl” refers to the divalent group —NH-alkanediyl-, —NH-alkanediyl-NH—, or -alkanediyl-NH-alkanediyl-. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The groups —NHC(O)OCH₃ and —NHC(O)NHCH₃ are non-limiting examples of substituted amido groups.

### B. Extracellular pHₑ

The present disclosure also relates to imaging the extracellular pH (pHₑ) of a cell or group of cells. In particular, the extracellular environment could be of a tumor cell. Aerobic glycolysis (a.k.a., Warburg effect), where cancer cells preferentially take up glucose and convert it into lactic acids, has rekindled intense interest in imaging pHₑ of a tumor cell as a method of determine the presence of tumor tissue (Heiden et al., 2009). The clinical relevance of the Warburg effect has already been manifested by the wide clinical use of 2-¹⁸F-deoxyglucose (FDG) for tumor diagnosis as well as monitoring treatment responses. In tumor microenvironment, lactic acids are preferentially accumulated in the extracellular space due to monocarboxylate transporters, which are elevated in cancer cell membranes (Halestrap & Prince 1999). The resulting acidification of extracellular pH (pHₑ) in tumors promotes remodeling of extracellular matrix for increased tumor invasion and metastasis. Recently, Barber and coworkers described dysregulated pH in tumors as another “hallmark of cancer” (Webb et al., 2011).

Many previous studies have been performed to quantify the pHₑ in the tumor microenvironment (Gillies et al., 1994; Gillies et al., 2004; van Sluis et al., 1999 and Volk et al., 1993), including a representative pHₑ study in 268 tumors from 30 different human cancer cell lines (Volk et al., 1993). Compared to blood pH (7.4), all the tumor pHₑ are acidic with an average of 6.84 ranging from 6.71 to 7.01. Although the acidity of tumor pHₑ is persistent, exploiting it for tumor-specific imaging is challenging due to the relatively small pH differences (i.e., <1 pH unit) making probes which possess a very narrow pH transition range of particular interest for this application.

In some embodiments, the present disclosure provides polymers and micelles which can be used in a pH responsive system that can image and physiological and/or pathological process that is affected or affects intracellular or extracellular pH including but not limited to infections, fistulas, ulcers, ketoacidosis from diabetes or other diseases, hypoxia, metabolic acidosis, respiratory acidosis, toxic ingestion, poisoning, bone turnover, degenerative diseases, wounds, and tissue damage from burns radiation or other sources.

### C. Surgical Imaging of Tumor Margins

Positive tumor margins, which are defined by the presence of cancer cells at the edge of surgical resection, are the most important indicator of tumor recurrence and survival of HNSCC patients after surgery (Woolgar & Triantafyllou 2005; McMahon et al., 2003; Ravasz et al., Atkins et al., 2012 and Iczkowski & Lucia 2011). In some embodiments, any cancer cell line which exhibits a different extracellular pH environment than the normal physiological pH of the environment can be imaged with a pH responsive system disclosed herein. Furthermore, by modifying the dye used in the pH responsive dyes, a variety of different commercially available surgical imaging systems can be used to measure the margins of the tumor. These systems include but are not limited to systems for open surgery (e.g., SPY Elite®), microsurgery (Carl Zeiss, Leica), laparoscopy (Olympus, Karl Storz), and robotic surgery (da Vinci®). Many of these clinical systems have fast acquisition times allowing real-time imaging during an operation. Furthermore, the mixed polymers disclosed herein as well as a homopolymer of the any of the individual monomers used to create the mixed polymers can be used in the pH responsive system for the imaging of a tumor during an operation.

### D. Block Copolymers and Fluorescent Dyes

The pH-responsive micelles and nanoparticles disclosed herein comprise block copolymers and fluorescent dyes. A block copolymer comprises a hydrophilic polymer segment and a hydrophobic polymer segment. The hydrophobic polymer segment is pH sensitive. For example, the hydrophobic polymer segment may comprise an ionizable amine group to render pH sensitivity. The block copolymers form pH-activatable micellar (pHAM) nanoparticles based on the supramolecular self-assembly of these ionizable block copolymers. At higher pH, the block copolymers assemble into micelles, whereas at lower pH, ionization of the amine group in the hydrophobic polymer segment results in dissociation of the micelle. The ionizable groups may act as tunable hydrophilic/hydrophobic blocks at different pH values, which may directly affect the dynamic self-assembly of micelles.

For diagnostic or pH monitoring applications, a labeling moiety may be conjugated to the block copolymer. In some embodiments, the label (e.g., a fluorescent label) is sequestered inside the micelle when the pH favors micelle formation. Sequestration in the micelle results in a decrease in label signal (e.g., via fluorescence quenching). Specific pH conditions may lead to rapid protonation and dissociation of micelles into unimers, thereby exposing the label, and increasing the label signal (e.g., increasing fluorescence emission). The micelles of the disclosure may provide one or more advantages in diagnostic applications, such as: (1) disassociation of the micelle (and rapid increase in label signal) within a short amount of time (e.g., within minutes) under certain pH environments (e.g., acidic environments), as opposed to hours or days for previous micelle compositions; (2) increased imaging payloads; (3) selective targeting of label to the desired site (e.g., tumor or particular endocytic compartment); (4) prolonged blood circulation times; (5) responsiveness within specific narrow pH ranges (e.g., for targeting of specific organelles); and (6) high contrast sensitivity and specificity. For example, the micelles may stay silent (or in the OFF state) with minimum background signals under normal physiological conditions (e.g., blood circulation, cell culture conditions), but imaging signals can be greatly amplified when the micelles reach their intended molecular targets (e.g., extracellular tumor environment or cellular organelle).

Numerous fluorescent dyes are known in the art. In certain aspects of the disclosure, the fluorescent dye is a pH-insensitive fluorescent dyes. In some embodiments, the fluorescent dye is paired with a fluorescent quencher to obtain an increased signal change upon activation. The fluorescent dye may be conjugated to the copolymer directly or through a linker moiety. Methods known in the art may be used to conjugate the fluorescent dye to, for example, the hydrophobic polymer. In some embodiments, the fluorescent dye may be conjugated to amine of the hydrophobic polymer through an amide bond.

Examples of block copolymers and block copolymers conjugated to fluorescent dyes and metal chelating groups include:

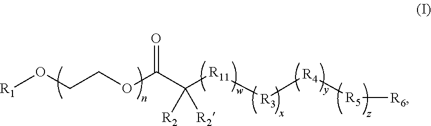

wherein: R₁ is hydrogen, alkyl_{(C≤12)}, cycloalkyl_{(C≤12)}, substituted alkyl_{(C≤12)}, substituted cycloalkyl_{(C≤12)}, or

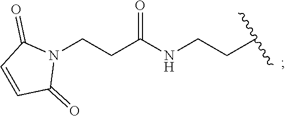 [SMILES: *CCNC(=O)CCN1C(=O)C=CC1=O]

n is an integer from 1 to 500; R₂ and R₂′ are each independently selected from hydrogen, alkyl_{(C≤12)}, cycloalkyl_{(C≤12)}, substituted alkyl_{(C≤12)}, or substituted cycloalkyl_{(C≤12)}; R₃ and R₁₁ are each independently a group of the formula:

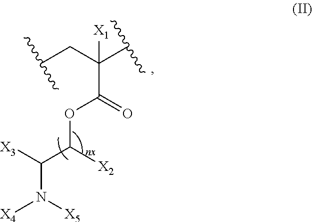

wherein: nₓ is 1-10; X₁, X₂, and X₃ are each independently selected from hydrogen, alkyl_{(C≤12)}, cycloalkyl_{(C≤12)}, substituted alkyl_{(C≤12)}, or substituted cycloalkyl_{(C≤12)}; and X₄ and X₅ are each independently selected from alkyl_{(C≤12)}, cycloalkyl_{(C≤12)}, aryl_{(C≤12)}, heteroaryl_{(C≤12)} or a substituted version of any of these groups, or X₄ and X₅ are taken together and are alkanediyl_{(C≤12)}, alkoxydiyl_{(C≤12)}, alkylaminodiyl_{(C≤12)}, or a substituted version of any of these groups; w is an integer from 0 to 150; x is an integer from 1 to 150; R₄ is a group of the formula:

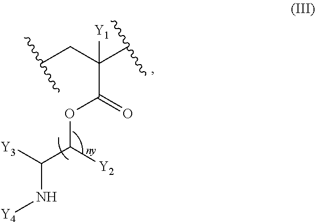

wherein: n_{y} is 1-10; Y₁, Y₂, and Y₃ are each independently selected from hydrogen, alkyl_{(C≤12)}, cycloalkyl_{(C≤12)}, substituted alkyl_{(C≤12)}, or substituted cycloalkyl_{(C≤12)}; and Y₄ is a dye or a fluorescence quencher; y is an integer from 1-6; R₅ is a group of the formula:

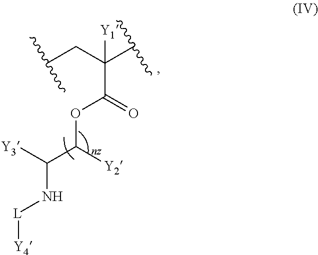

wherein: n_{z} is 1-10; Y₁′, Y₂′, and Y₃′ are each independently selected from hydrogen, alkyl_{(C≤12)}, cycloalkyl_{(C≤12)}, substituted alkyl_{(C≤12)}, or substituted cycloalkyl_{(C≤12)}; and Y₄′ is a metal chelating group; L is a covalent bond; or alkanediyl_{(C≤12)}, arenediyl_{(C≤12)}, -alkanediyl_{(C≤12)}-arenediyl_{(C≤12)}—NC(S)—, -alkanediyl_{(C≤12)}-arenediyl_{(C≤12)}-C(O)—, or a substituted version of any of these groups; z is an integer from 1-6; and R₆ is hydrogen, halo, hydroxy, alkyl_{(C≤12)}, or substituted alkyl_{(C≤12)}, wherein R₁₁, R₃, R₄, and R₅ can occur in any order within the polymer. In some embodiments, each monomer of R₁₁, R₃, R₄, and R₅ within the longer polymer can occur in any order within the polymer. In some embodiments, the specific composition of the polymer (molar fraction of each R₃, R₄, and R₅ monomers) is related to the specific pH transition point of the nanoparticle produced using that polymer.

### E. Micelle Systems and Compositions

The systems and compositions disclosed herein utilize either a single micelle or a series of micelles tuned to different pH levels. Furthermore, the micelles have a narrow pH transition range. In some embodiments, the micelles have a pH transition range of less than about 1 pH unit. In various embodiments, the micelles have a pH transition range of less than about 0.9, less than about 0.8, less than about 0.7, less than about 0.6, less than about 0.5, less than about 0.4, less than about 0.3, less than about 0.25, less than about 0.2, or less than about 0.1 pH unit. The narrow pH transition range advantageously provides a sharper pH response that can result in complete turn-on of the fluorophores with subtle changes of pH.

Accordingly, a single or series of pH-tunable, multicolored fluorescent nanoparticles having pH-induced micellization and quenching of fluorophores in the micelle core provide mechanisms for the independent control of pH transition (via polymers), fluorescence emission, or the use of fluorescence quenchers. The fluorescence wavelengths can be fine-tuned from, for example, violet to near IR emission range (400-820 nm). Their fluorescence ON/OFF activation can be achieved within no more than 0.25 pH units, which is much narrower compared to small molecular pH sensors. In some embodiments, a narrower range for fluorescence ON/OFF activation can be achieved such that the range is no more than 0.2 pH units. In some embodiments, the range is no more than 0.15 pH units. Furthermore, the use of a fluorescence quencher may also increase the fluorescence activation such that the difference between the associated and disassociated nanoparticle is greater than 50 times the associated nanoparticle. In some embodiments, the fluorescence activation is greater than 75 times higher than the associated nanoparticle This multicolored, pH tunable and activatable fluorescent nanoplatform provides a valuable tool to investigate fundamental cell physiological processes such as pH regulation in endocytic organelles, receptor cycling, and endocytic trafficking, which are related to cancer, lysosomal storage disease, and neurological disorders.

The size of the micelles will typically be in the nanometer scale (i.e., between about 1 nm and 1 μm in diameter). In some embodiments, the micelle has a size of about 10 to about 200 nm. In some embodiments, the micelle has a size of about 20 to about 100 nm. In some embodiments, the micelle has a size of about 30 to about 50 nm.

### F. Targeting Moieties

The micelles and nanoparticles may further comprise a targeting moiety. The targeting moiety may be used to target the nanoparticle or micelle to, for example, a particular cell surface receptor, cell surface marker, or to an organelle (e.g., nucleus, mitochondria, endoplasmic reticulum, chloroplast, apoplast, or peroxisome). Such targeting moieties will be advantageous in the study of receptor recycling, marker recycling, intracellular pH regulation, endocytic trafficking.

The targeting moiety may be, for example, an antibody or antibody fragment (e.g., Fab′ fragment), a protein, a peptide (e.g., a signal peptide), an aptamer, or a small molecule (e.g., folic acid). The targeting moiety may be conjugated to the block copolymer (e.g., conjugated to the hydrophilic polymer segment) by methods known in the art. The selection of targeting moiety will depend on the particular target. For example, antibodies, antibody fragments, small molecules, or binding partners may be more appropriate for targeting cell surface receptors and cell surface markers, whereas peptides, particularly signal peptides, may be more appropriate for targeting organelles.

### G. Fluorescence Detection

Various aspects of the present disclosure relate to the direct or indirect detection of micelle disassociation by detecting an increase in a fluorescent signal. Techniques for detecting fluorescent signals from fluorescent dyes are known to those in the art. For example, fluorescence confocal microscopy as described in the Examples below is one such technique.

Flow cytometry, for example, is another technique that can be used for detecting fluorescent signals. Flow cytometry involves the separation of cells or other particles, such as microspheres, in a liquid sample. The basic steps of flow cytometry involve the direction of a fluid sample through an apparatus such that a liquid stream passes through a sensing region. The particles should pass one at a time by the sensor and may categorized based on size, refraction, light scattering, opacity, roughness, shape, fluorescence, etc.

The measurements described herein may include image processing for analyzing one or more images of cells to determine one or more characteristics of the cells such as numerical values representing the magnitude of fluorescence emission at multiple detection wavelengths and/or at multiple time points.

### H. Kits

The present disclosure also provides kits. Any of the components disclosed herein may be combined in a kit. In certain embodiments the kits comprise a pH-responsive system or composition as described above.

The kits will generally include at least one vial, test tube, flask, bottle, syringe or other container, into which a component may be placed, and preferably, suitably aliquoted. Where there is more than one component in the kit, the kit also will generally contain a second, third or other additional containers into which the additional components may be separately placed. However, various combinations of components may be comprised in a container. In some embodiments, all of the micelle populations in a series are combined in a single container. In other embodiments, some or all of the micelle population in a series are provided in separate containers.

The kits of the present disclosure also will typically include packaging for containing the various containers in close confinement for commercial sale. Such packaging may include cardboard or injection or blow molded plastic packaging into which the desired containers are retained. A kit may also include instructions for employing the kit components. Instructions may include variations that can be implemented.

### I. SPECT and PET

Radionuclide imaging modalities (positron emission tomography, (PET); single photon emission computed tomography (SPECT)) are diagnostic cross-sectional imaging techniques that map the location and concentration of radionuclide-labeled radiotracers. Although CT and MRI provide considerable anatomic information about the location and the extent of tumors, these imaging modalities cannot adequately differentiate invasive lesions from edema, radiation necrosis, grading or gliosis. PET and SPECT can be used to localize and characterize tumors by measuring metabolic activity.

PET and SPECT provide information pertaining to information at the cellular level, such as cellular viability. In PET, a patient ingests or is injected with a slightly radioactive substance that emits positrons, which can be monitored as the substance moves through the body. In one common application, for instance, patients are given glucose with positron emitters attached, and their brains are monitored as they perform various tasks. Since the brain uses glucose as it works, a PET image shows where brain activity is high.

Closely related to PET is single-photon emission computed tomography, or SPECT. The major difference between the two is that instead of a positron-emitting substance, SPECT uses a radioactive tracer that emits low-energy photons. SPECT is valuable for diagnosing coronary artery disease, and already some 2.5 million SPECT heart studies are done in the United States each year.

PET radiopharmaceuticals for imaging are commonly labeled with positron-emitters such as ¹¹C, ¹³N, ¹⁵O, ¹⁸F, ⁸²Rb, ⁶²Cu, and ⁶⁸Ga. SPECT radiopharmaceuticals are commonly labeled with positron emitters such as ^{99m}Tc, ²⁰¹Tl, and ⁶⁷Ga. Regarding brain imaging, PET and SPECT radiopharmaceuticals are classified according to blood-brain-barrier permeability (BBB), cerebral perfusion and metabolism receptor-binding, and antigen-antibody binding (Saha et al., 1994). The blood-brain-barrier SPECT agents, such as ⁹⁹TcO4-DTPA, ²⁰¹Tl, and [⁶⁷Ga]citrate are excluded by normal brain cells, but enter into tumor cells because of altered BBB. SPECT perfusion agents such as [¹²³I]IMP, [^{99m}Tc]HMPAO, [^{99m}Tc]ECD are lipophilic agents, and therefore diffuse into the normal brain. Important receptor-binding SPECT radiopharmaceuticals include [¹²³I]QNE, [¹²³I]IBZM, and [¹²³I]iomazenil. These tracers bind to specific receptors and are of importance in the evaluation of receptor-related diseases.

### J. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Synthesis and Evaluation of Dual Modality pH Responsive Nanoprobes

A. Synthesis of ⁶⁴Cu-UPS_{6.9}

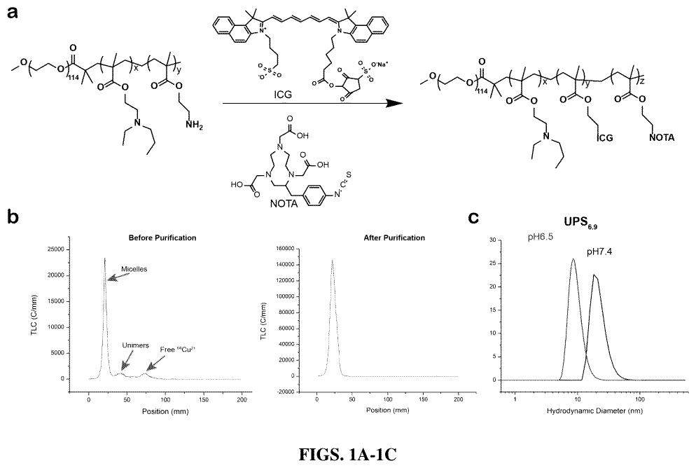

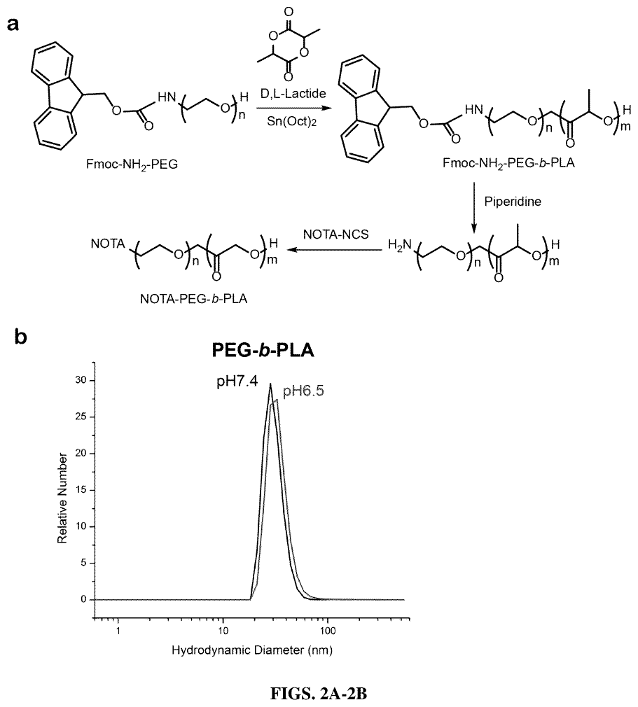

1,4,7-Triazacyclononane-N,N′,N″-trisacetic acid (NOTA)- and ICG-conjugated poly(ethylene glycol)-b-poly(ethylpropylaminoethyl methacrylate) copolymer (aka UPS_{6.9} for pH transition at 6.9) was synthesized by the atom transfer radical polymerization method (FIG. 1A; Tsarevsky et al., 2007). The average numbers of NOTA and ICG per copolymer were determined to be 2 and 1, respectively. After polymer synthesis, ⁶⁴Cu chelation to NOTA was carried out at 37° C. and pH 6.5 for 15 mins to ensure fully dissociated unimers for efficient copper binding (95%, FIG. 1B). After ⁶⁴Cu labeling, the solution was brought back to pH 7.4 in sodium carbonate buffer to form micelle nanoparticles (32.7±1.6 nm) (FIG. 1C). Removal of unbound ⁶⁴CuCl₂ was achieved by centrifugal membrane filtration three times with a molecular weight cutoff of 100 kD. Lowering the pH below the transition pH led to micelle dissociation into unimers (8.4±0.2 nm). For comparison, NOTA-conjugated poly(ethylene glycol)-b-poly(D,L-lactic acid) (PEG-PLA) nanoparticles with similar size (32.0±2.4 nm) were also synthesized as a non-pH sensitive nanosensor control (FIG. 2). Additional UPS compounds comprising different alkylated amino moieties in addition to a dye or fluorescence metal chelating groups were prepared and their pH transitions evaluated and recorded as a function of the percentage of each type of monomer present in the polymer. For example, varying the proportion of monomers comprising diethylamino moieties and monomers comprising diisopropylamino moieties in a UPS polymer modulated the pH transition and allowed the pH transition to be fine-tuned (FIGS. 17A-C).

B. Irreversible Activation of ⁶⁴Cu-UPS_{6.9} in Biological Milieu

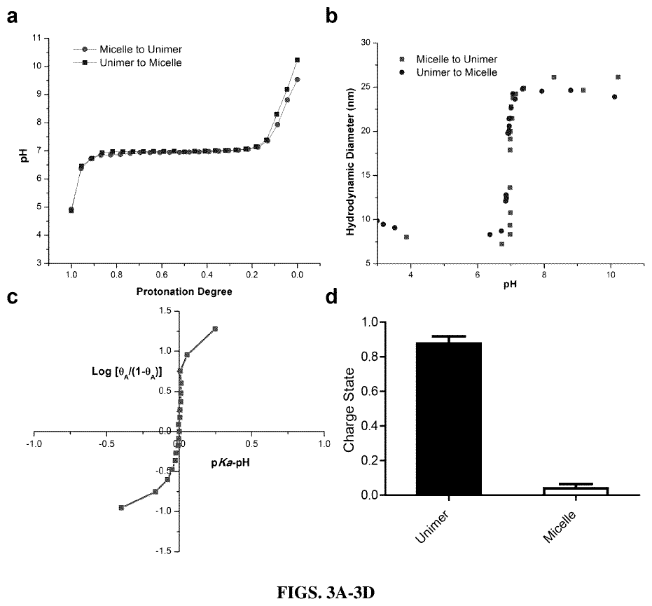

In aqueous saline solution (protein free), UPS_{6.9} copolymers undergo “reversible” micelle assembly/disassembly across a narrow pH span (<0.2 pH, FIGS. 3A & 3B). The protonation process is highly cooperative with a Hill coefficient of 38 (FIG. 3C). Along the pH titration coordinate, phase segregation (i.e., micellization) rendered a bistable state solution consisting of highly protonated unimers in solution versus neutral copolymers in micelles (FIG. 3D). This all-or-nothing protonation phenotype without the intermediate states is a hallmark of positive cooperativity (Lopez-Fontal et al., 2016 and Williamson, 2008). The divergent physical properties of the neutral PEGylated micelles and polycationic unimers account for the molecular basis of capture and integration mechanism in the biological system.

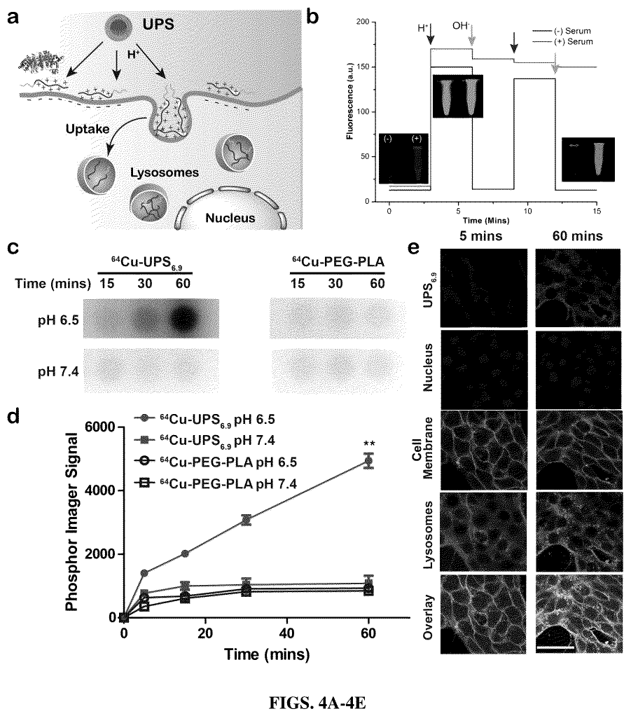

In biological milieu, serum protein binding can “irreversibly” arrest UPS copolymers in the dissociated unimer state upon pH activation of PEGylated micelles (FIG. 4A). The reversibility of the UPS_{6.9} nanoprobes in the presence or absence of 40 mg/ml human serum albumin (HSA) was examined (FIG. 4B). Results show in the absence of HSA, UPS_{6.9} fluorescence intensity was returning to the base level after pH is reversed from 6.5 to 7.4 multiple times. In contrast, in the presence of HSA, the fluorescence intensity was kept at the on state after pH reversal. These data suggest that nanoprobe response can be drastically different in the biological environment compared to pristine buffer solutions. This irreversibility characteristic contributes to the capture of persistent but fluctuating tumor acidotic signals into stabilized output.

C. Irreversible Capture and Uptake of ⁶⁴Cu-UPS_{6.9} by Cancer Cells

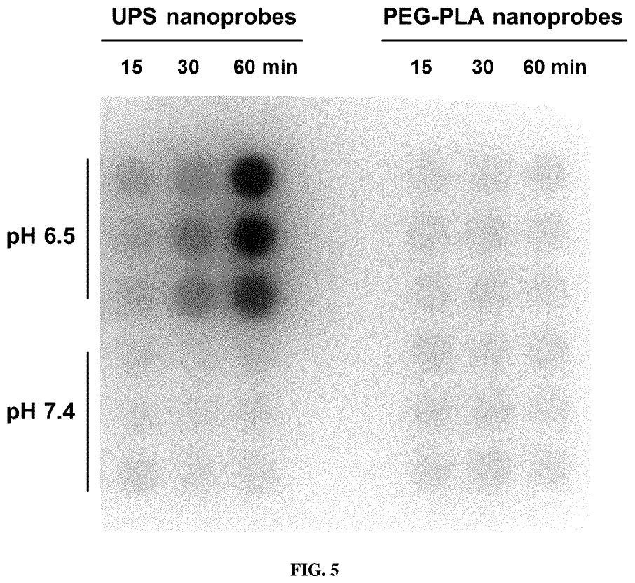

To investigate whether acidic pH can impact nanoprobe uptake inside cancer cells, ⁶⁴Cu-UPS_{6.9} was incubated with HN5 head and neck cancer cells in DMEM medium at pH 6.5 and 7.4. To mimic the physiological environment, 40 mg/mL human serum albumin was added in the medium. For comparison, ⁶⁴Cu-PEG-PLA nanoparticles were used as a non-pH sensitive control. HN5 cells were incubated with the same dose of either nanoparticles (25 μg/mL) for different time periods. Then the radioactive medium was replaced and washed with medium without the tracers. Autoradiography images of HN5 cells from the 96-well plate show pH-dependent uptake of ⁶⁴Cu-UPS_{6.9}. At pH 6.5, increased amount of positron signals was detected over time, leading to an approximately 5-fold increase in cell uptake at 1 h over that at pH 7.4 (FIGS. 4C and 4D, and FIG. 5). In contrast, HN5 cells incubated with ⁶⁴Cu-PEG-PLA micelles did not show any observable pH dependence in radioactivity signals, and the cell uptake remained low at both pH, consistent with the stealth properties of PEGylated micelle nanoparticles (Moghimi et al., 2003). Laser confocal scanning microscopy was used to examine the distribution of the UPS_{6.9} (free of ⁶⁴Cu to avoid radiation exposure) 5 and 60 mins after incubation in albumin-containing medium at pH 6.5. HN5 cells were stained for nucleus, cell membrane and lysosomes by Hoechst (blue), anti-F-117 actin (cyan) and anti-LAMP1 (green), respectively. Anti-poly(ethylene glycol) antibody was used to label the UPS_{6.9} copolymer. Data show the initial adhesion of the copolymer on the cell surface, followed by internalization inside the HN5 cells at 60 mins. Image overlay shows the internalized UPS_{6.9} punctates colocalized with lysosomes (FIG. 4E).

D. In Vivo Capture and Integration of ⁶⁴Cu-UPS_{6.9} in the HN5 Tumors

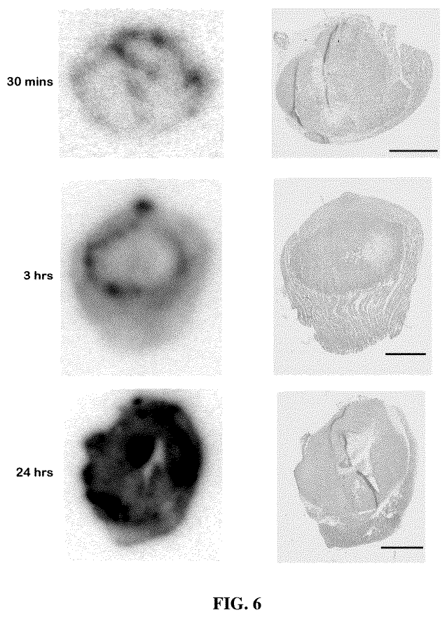

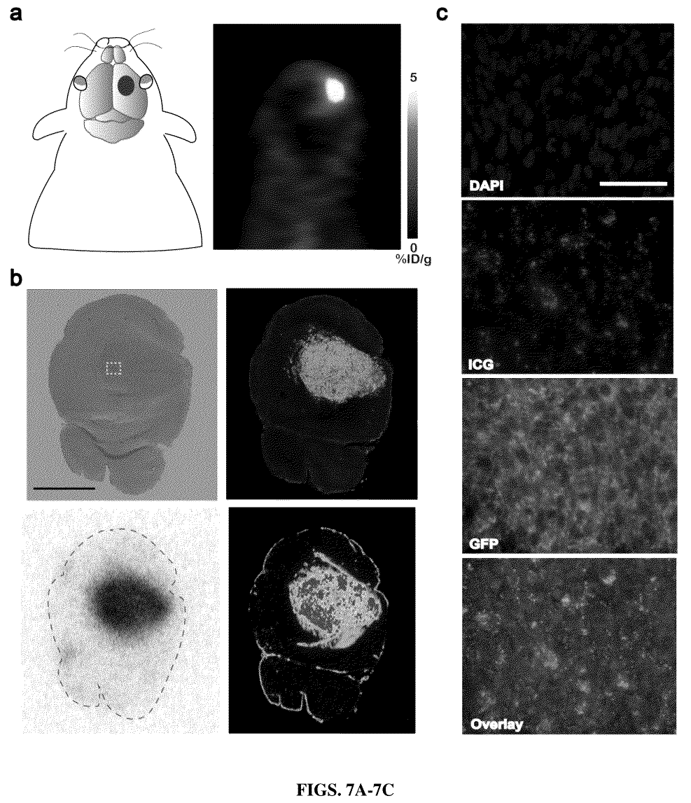

Spatio-temporal characterization of ⁶⁴Cu-UPS_{6.9} distribution in orthotopic HN5 tumors in vivo further validated the capture and integration mechanism. HN5 cancer cells were inoculated in the submental space in the head and neck area of a SCID mouse. After tumors grew to 20-30 mm³, ⁶⁴Cu-UPS_{6.9} tracer (0.1 mCi) was injected through the tail vein. At 30 mins, 3 h, and 24 h, animals were sacrificed, and tumors were removed and resected into 30 μm thin slices. Autoradiography analysis showed the initial sporadic capture of ⁶⁴Cu-UPS_{6.9} in HN5 tumors (mostly at the tumor periphery) at 30 mins and 3 has verified by H&E histology, followed by increased tracer accumulation throughout the whole tumor at 24 h (FIG. 6).

E. ⁶⁴Cu-UPS_{6.9} Achieved Binary Detection of Brain Tumors

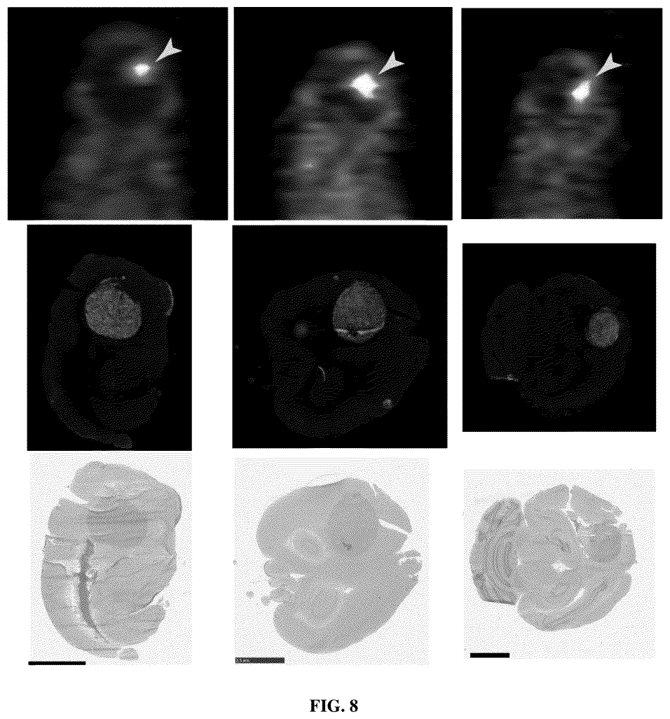

Brain cancer is one of the most lethal forms of cancer without a widely accepted method for early detection (Wen and Kesari, 2008). Late diagnosis when symptoms occur often leads to poor prognosis and survival (Omuro and DeAngelis, 2013). Conventional metabolic PET tracer FDG cannot be used for brain tumor imaging because of the high physiologic uptake of glucose in the normal brain tissues (Fink et al., 2015). To investigate the feasibility of ⁶⁴Cu-UPS_{6.9} for glioma detection, evaluation of an orthotopic tumor xenograft model transplanted with green fluorescent protein (GFP)-transfected murine astrocyte with p53^{-/-}, PTEN^{-/-}, BRAF^{V600E} mutations (73C) was undertaken. At 24 h after intravenous administration of ⁶⁴Cu-UPS_{6.9}, PET imaging showed a bright illumination of small sized brain tumors (˜10 mm³) over the dark normal brain tissue background (FIG. 7A and FIG. 8). Tissue uptake of ⁶⁴Cu-UPS_{6.9} was measured at 3.1±1.6 and 0.54±0.3% ID/g for 73C tumors and normal brain tissues, respectively. The contrast over noise ratio (CNR, which is calculated as the difference in signal intensity between tumor and normal tissue divided by the background noise) was determined to be 15.1±6.8. In the normal brain tissues, blood-brain barrier was effective at keeping the PEGylated micelle form of ⁶⁴Cu-UPS_{6.9} out of the brain parenchyma. In contrast, tumor acidosis was able to activate ⁶⁴Cu-UPS_{6.9}, leading to significantly increased positron signals. Subsequent investigation of brain tumor slides (8 μm in thickness) by autoradiography and indocyanine green fluorescence correlated microscopically with H&E histology and GFP fluorescence (FIG. 7B). Confocal microscopy data illustrates that the UPS tracer can cross the blood brain barrier in 73C gliomas and accumulate in many GFP-labeled brain cancer cells (overlay image in FIG. 7C). These data corroborate the feasibility of irreversibly trapping UPS tracers in brain cancer cells to achieve binary tumor imaging outcomes at both macroscopic and microscopic levels.

F. Non-Invasive Imaging of Multiple Tumor Types by ⁶⁴Cu-UPS_{6.9}

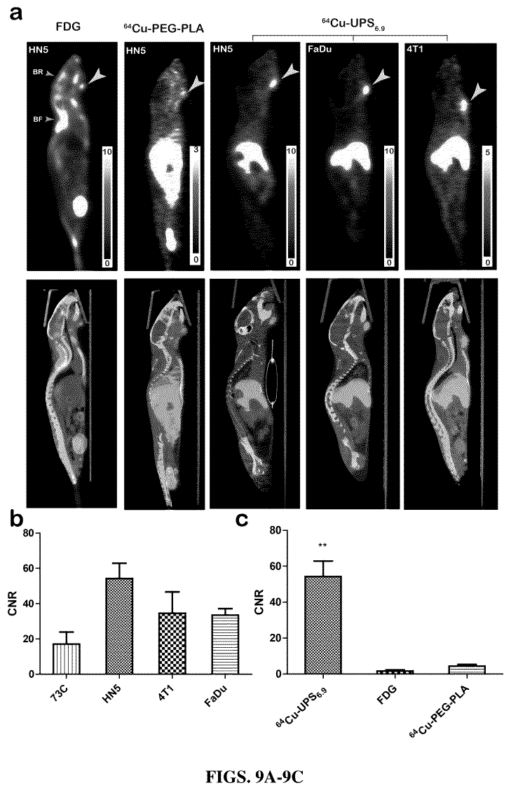

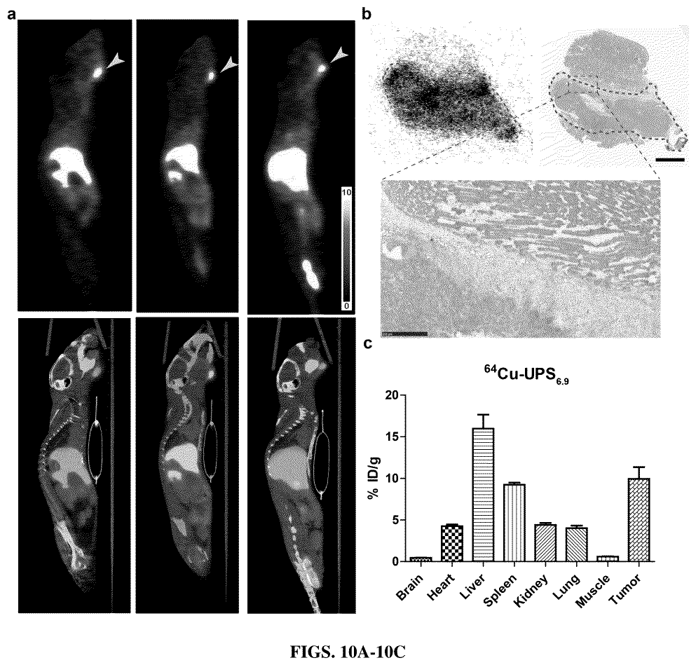

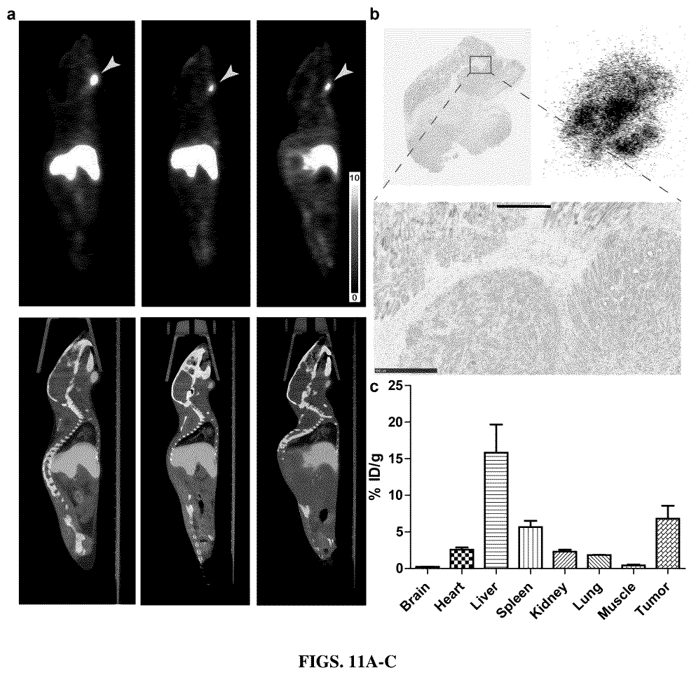

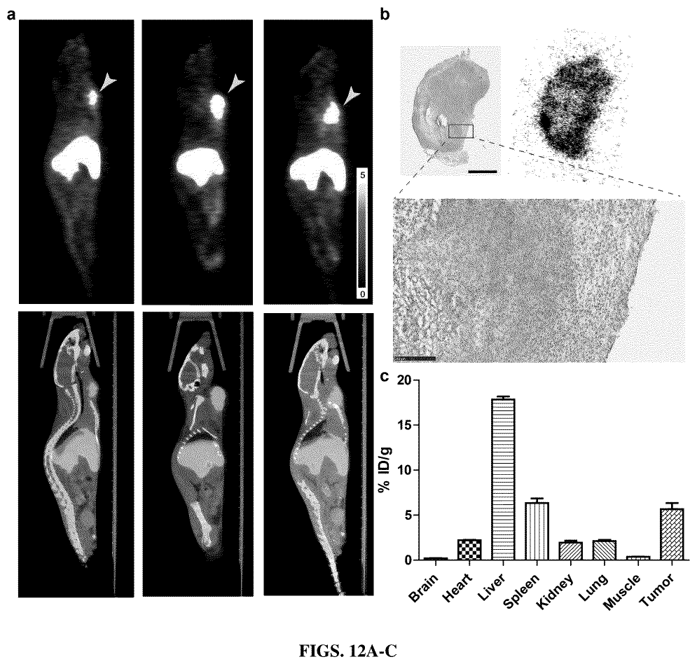

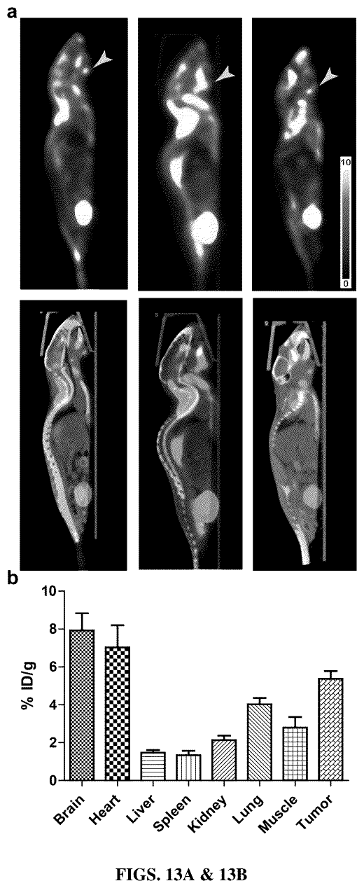

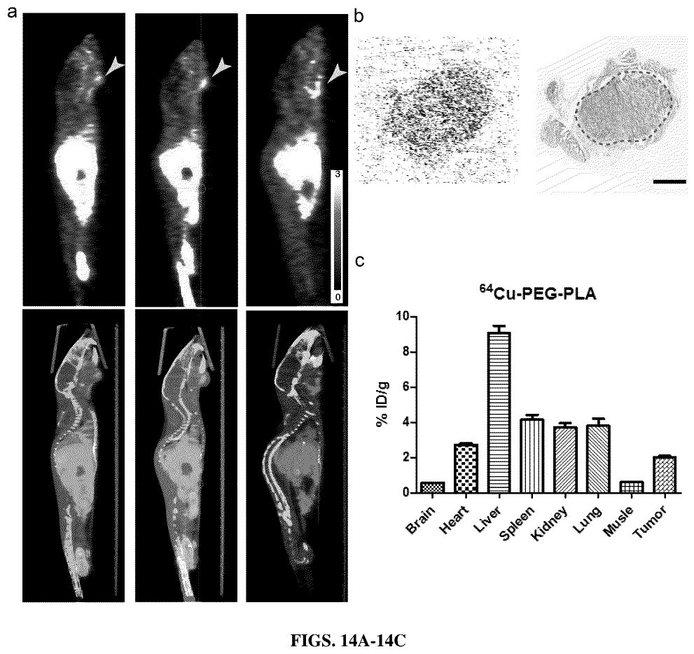

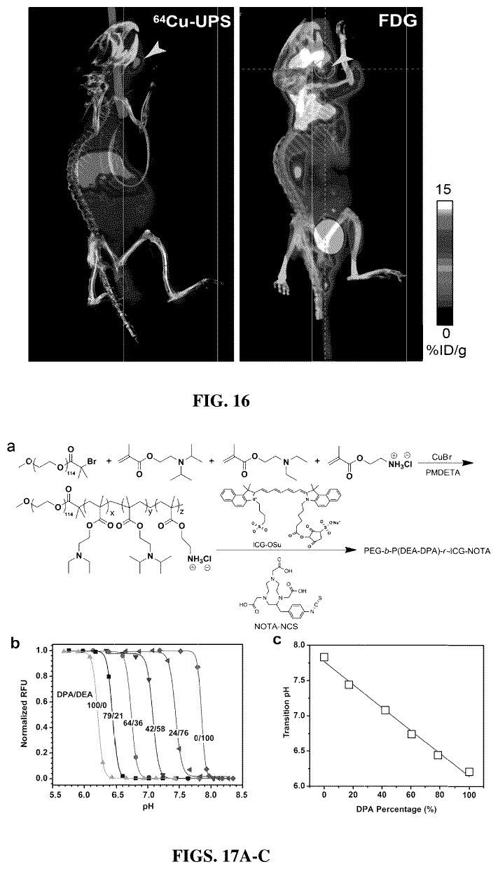

To investigate the feasibility of ⁶⁴Cu-UPS_{6.9} to image a broad set of cancers, PET imaging of additional head/neck and breast tumor nodules were evaluated. ⁶⁴Cu-UPS_{6.9} tracers (0.1 mCi) were injected in the tail vein of tumor-bearing mice. Results show conspicuous detection of occult nodules (10-20 mm³) in orthotopic HN5 and FaDu head and neck tumors, as well as 4T1 triple negative breast tumors (FIG. 9A and FIGS. 10-12 for triplicate reports demonstrating robustness of tumor detection). The tissue uptake was 9.9±2.5, 6.5±2.5 and 5.7±1.2% ID/g in the HN5, FaDu and 4T1 tumors 18-24 h after i.v. injection of ⁶⁴Cu-UPS_{6.9} tracers, respectively. The contrast over noise ratios were 54.3±8.7, 33.5±3.7 and 34.6±12.1 in the HN5, FaDu and 4T1 tumors (n=3 for each tumor type), respectively (FIG. 9B). PET imaging using FDG (0.15 mCi) and ⁶⁴Cu-PEG-PLA (0.12 mCi) in HN5 tumors showed less striking imaging outcomes. In FDG-PET experiments, despite HN5 tumor contrast (5.4±0.7% ID/g), non-specific uptake of the FDG in brain (7.9±1.6% ID/g), brown fats (8.1±1.3% ID/g), tensed muscles (6.3±0.3% ID/g) created false positive signals that complicated tumor diagnosis (FIGS. 9A & 9C, FIG. 13, and FIG. 16). The high background noise also decreased the CNR value to 1.7±0.6. In the ⁶⁴Cu-PEG-PLA study, a small percentage (2.0±0.2% ID/g) of tumor uptake was observed in HN5 tumors 18-24 h after i.v. injection. The CNR value of ⁶⁴Cu-PEG-PLA (4.4±1.0, FIG. 9C and FIG. 14) is significantly lower than ⁶⁴Cu-UPS_{6.9} (54.3±8.7). These results demonstrate that passive targeting through the leaky tumor vasculature is not sufficient to produce high tumor contrast as shown by the low CNR value of conventional ⁶⁴Cu-PEG-PLA micelle probes.

G. Discussion

Biological processes are dynamic and complex with perpetual changes in space and time. The resulting spatio-temporal heterogeneity makes it challenging to accurately diagnose pathologic conditions. Previously an ICG-based ultra-pH sensitive (UPS) nanoprobe was reported for cancer detection by fluorescence imaging (Zhao et al., 2016). A binary fluorescent delineation of tumor margins was achieved, which led to accurate cancer surgery and prolonged survival in tumor-bearing mice. The main mechanism was thought to be the coupling of the pH dependent phase transition phenomenon to the quenching and unquenching of the fluorophores conjugated to the hydrophobic segment of the polymers. In this present disclosure, a ⁶⁴Cu PET functional moiety was incorporated into the fluorescent nanoparticle formulation. In particular, the PET functional moiety was also conjugated to the hydrophobic segment of the polymers. Unlike the ON/OFF fluorescence reporter, the positron signals are always ‘ON’ and cannot be quenched, therefore phase transition-based changes in signal analogous to fluorescence were not expected. Contrary to expectation, the positron signal showed a binary pattern of background signal suppression and tumor activation similar to the fluorescence output (FIG. 7 and FIG. 9). While this overcame the light penetration limitations of optical imaging, the mechanisms for the unpredicted pattern of the positron signal was of curiosity. Clearly, passive accumulation due to EPR effect alone was not sufficient to produce the high tumor contrast, as indicated by the relatively low CNR value of ⁶⁴Cu-PEG-PLA compared to ⁶⁴Cu-UPS in HN5 tumors.

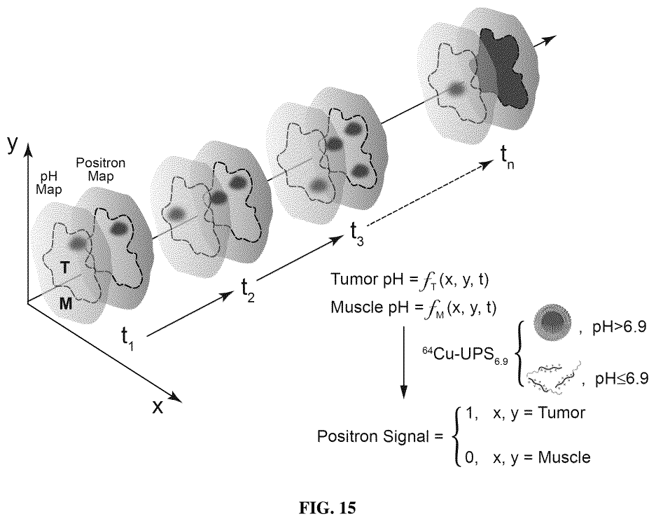

Without wishing to be bound by theory, the improved sensitivity and specificity of cancer detection by ⁶⁴Cu-UPS tracers is believed to be attributed to a “capture and integration” mechanism in the acidotic tumor milieu (FIG. 15). Like most biological signals, tumor acidosis is dynamic with high intratumoral heterogeneity in space and time. Reversible small molecular pH sensors (Gillies et al., 1994 and Gillies et al., 2004) do not show high tumor contrast due to broad pH response leading to background activation and incomplete tumor activation. In addition, their signal output varies with the transient fluctuations in tumor metabolism and pH. In comparison, binary and irreversible activation of UPS nanoprobes below a threshold pH (e.g., 6.9, readily achievable by a diverse set of tumors (Volk et al., 1993) can permanently convert the spatio-temporal fluctuations of tumor acidotic signals into stable, binary positron output. The sharpness of the phase transition response in this instance is converted into the specific retention or capture of the ⁶⁴Cu-bearing polymers in acidic tissues such as tumors, while capture is suppressed in the background normal tissue. More specifically, at different time points (t₁, t₂, . . . tₙ), different regions of the tumor can be acidified below the pH threshold (6.9) as indicated by the green spots in front images (FIG. 15). This transient acidotic signal in turn activates ⁶⁴Cu-UPS micelles circulating at the tumor site into polycationic unimers, which are irreversibly captured leaving a stable imprint of polymer signal (red spots in the back images). The irreversible capture resulted in increased dose accumulation over time for ⁶⁴Cu-UPS as validated experimentally (FIG. 6). Furthermore, arrest of polymers inside the lysosomes of cancer cells avoids diffusion-caused signal blurring, which may explain the sharp contrast at the tumor and normal tissue boundary even after 24 hrs. Intact micelles are cleared from the tumor sites as well as normal tissues through blood circulation. ⁶⁴Cu-UPS, by linking the binary activation in response to pH to a novel tissue retention output, suppresses the background while allowing maximal amplification of the tumor signal as approximated by 1 (tumor) or 0 (muscle/brain) outputs. Data show ⁶⁴Cu-UPS tracers were able to detect a broad range of occult cancer types in different anatomical sites (FIG. 7 and FIG. 9) including in the brain, head and neck where FDG imaging is typically obscured by the high signal found in normal brain and tonsil tissues. As stated earlier, FDG also employs a capture and integration mechanism to increase tumor contrast by the FDG uptake through the glucose transporters and arrest in the cancer cells after phosphorylation by hexokinase. Unlike ⁶⁴Cu-UPS, however, the process is reversible and not binary with background suppression. Although respectable dose accumulation of FDG was observed in tumors (e.g., 5.4±0.7% ID/g in HN5 tumors), high physiologic uptake of FDG in healthy tissues (e.g., 7.9±1.6, 8.1±1.3% and 6.3±0.3% ID/g in brain, brown fat and striated muscle, respectively) hampers cancer-specific detection of tumors. For ⁶⁴Cu-UPS, coupling the unique binary output of phase transition to capture of the acidotic signal allows a more cancer-specific detection of occult diseases (FIG. 9). Besides tumor acidosis, other factors such as leaky tumor vasculature, disrupted blood brain barriers (as in the case of 73C glioma detection), elevated micropinocytosis, and impaired lymphatics may also contribute to the robust contrast of tumors over surrounding normal tissues by ⁶⁴Cu-UPS. Meanwhile, high uptake of ⁶⁴Cu-UPS in the reticuloendothelial systems (e.g., liver and spleen) may preclude the use of this agent in the detection of cancers in these organs.

In summary, the molecular mechanism was determined for pH (proton) transistor-like nanoparticles to capture and integrate tumor acidotic signals into discrete outputs to improve the precision of cancer detection. This represents a second output, tissue retention, coupled to the transistor-like binary behavior of the UPS nanoparticles. The impact of the concept is illustrated by the non-invasive detection of small occult diseases (10-20 mm³ or 3-4 mm) in the brain, head and neck, and breast by PET imaging. Incorporation of both PET and fluorescence functions in one UPS nanoplatform further synergizes two orthogonal imaging modalities, which potentially allow initial whole-body assessment of tumor burden by PET, followed by high resolution fluorescence imaging for local interventions (e.g., biopsy or surgery). Besides diagnostic imaging, this second output for the UPS technology in binary dose retention may also offer therapeutic benefit in tumor-targeted delivery of radionuclides (e.g., ¹⁷⁷Lu and ⁹⁰Y) or drugs with increased area under the curve (AUC). It is anticipated that the proposed chemical integration algorithm will immediately impact early cancer detection and surveillance while creating strategic insights to incorporate molecular cooperativity principles (Li et al., 2018) for the design of precision medicine.

### Example 2: Methods of Synthesis and Additional Data

A. Synthesis of ICG- and NOTA-Conjugated UPS_{6.9} Nanoprobes and NOTA-PEG-b-PLA Nanoparticles

Poly(ethylene glycol)-b-poly(ethylpropylaminoethyl methacrylate) (PEG-b-PEPA) copolymer was synthesized following the reported procedure using the atom transfer radical polymerization method (Zhao et al., 2016). The polymers were then dissolved in methanol, ICG-Sulfo-OSu were first added to react with AMA (1:1 molar ratio) through via NHS-ester chemistry (Ma et al., 2014) for 1 hour. Next, p-SCN-Bn-NOTA were added to react with the remaining AMA (4:1 molar ratio) overnight at room temperature. Unconjugated ICG and NOTA were removed by Millipore ultrafiltration membranes with a molecular weight cutoff at 10 kDa. The UPS_{6.9} nanoprobes were produced by a solvent evaporation method (Wang et al., 2014) and concentrated to 5 mg/ml for further usage.

NOTA conjugated PEG-b-PLA block copolymer was synthesized by ring-opening polymerization following a published procedure (Blanco et al., 2010). Briefly, polymerization of D,L-lactide was performed at 110° C. using Fmoc-amine-PEG5K-hydroxyl as the macroinitiator and Sn(Oct)₂ as a catalyst. Deprotection of Fmoc was made by 20% piperidine in DMF. After polymer purification with precipitating in ether for three times, the solid polymer was suspended in DMF and reacted withp-SCN-Bn-NOTA at room temperature overnight. Unconjugated NOTA was removed by Millipore ultrafiltration membranes with a molecular weight cutoff at 10 kDa.

B. ⁶⁴Cu Labeling of UPS_{6.9} or PEG-b-PLA Nanoprobes

Chelation of ⁶⁴Cu²⁺ to NOTA on the UPS_{6.9} or PEG-b-PLA copolymer was accomplished by adjusting pH to 6.0-6.5 with 4 M ammonium acetate buffer for 15 mins at 37° C. Micelle formation was carried out by adjusting the solution pH to 7.4 with 2 M sodium carbonate. Removal of unbound ⁶⁴CuCl₂ was achieved by centrifugal membrane filtration with a molecular weight cutoff of 100 kD for three times. Before and after centrifugal filtration, 1 μL of micelle solution was mixed with 8 μL DI H₂O and 1 μL of 50 mM diethylenetriamine pentaacetate (DTPA) for 5 minutes. A 2 μL aliquot of the mixture was then spotted on a TLC plate and eluted with the mobile phase (PBS). The labeling efficiency was determined by radio-TLC.

C. pH Titration and Dialysis

UPS_{6.9} polymers were first dissolved in 2.5 mL 0.1 M HCl and diluted to 2.0 mg/mL with DI water. Sodium chloride was added to adjust the salt concentration to 150 mM. pH titration was performed by adding small volumes (1 μL in increments) of 4.0 M NaOH solution with stirring. The pH increase through the range of 3-11 was monitored as a function of total added volume of NaOH. The fully protonated state and complete deprotonation states (protonation degree equaled 100 and 0%, respectively) were determined by the two extreme value points of pH titration curves' 1st derivation. The pH values were measured using a Mettler Toledo pH meter with a microelectrode. Next, UPS_{6.9} polymers with protonation degree at 50% were obtained by adding corresponding volumes of 4.0 M NaOH. 10 mL of polymer solution was centrifuged using ultra-centrifugation tube with a molecular weight cutoff at 100 kDa to ˜5 mL filtrated sample. pH titrations were performed to quantify the amount of polymer and degree of protonation in both residual and filtrate layers. The experiments were repeated three times and the data shown is in mean±s.d.

D. Cell Culture

The cancer cell lines used for in vivo tumor models include HN5, FaDu, human head and neck cancers, 4T1 breast cancers, primary murine astrocyte cells with p53^{-/-}, PTEN^{-/-}, and BRAF^{V600E} mutation (73C). HN5 and FaDu cell lines were obtained from Michael Story's lab; 4T1 were obtained from the David Boothman lab; 73C was obtained from the Woo-Ping Ge lab. All cells lines were tested for mycoplasma contamination before use. Negative status for contamination was verified by Mycoplasma Detection Kit from Biotool. Cells were cultured in DMEM or RPMI with 10% fetal bovine serum and antibiotics.

E. Animal Models

Animal protocols related to this study were reviewed and approved by the Institutional Animal Care and Use Committee. Female NOD-SCID mice (6-8 weeks) were purchased from UT Southwestern Medical Center Breeding Core. For orthotopic head and neck tumors, HN5 and FaDu (2×10⁶ per mouse) were injected into the submental triangle area. One week after inoculation, animals with tumor size 20-100 mm³ were used for imaging studies. Orthotopic murine 4T1 breast tumor model was established in BalB/C mice by injection of 4T1 (5×10⁵ per mouse) cells into the mammary fat pads. GFP-transfected 73C murine glioblastoma tumor model was established by implanting 73C glioma cells intracranially in the left hemisphere of mice. Gliomas (2-4 mm in diameter) were formed within two weeks in mice.

F. Cell Uptake Assay

1.5×10⁴ HN5 cancer cells were seeded into individual well of 96-well plates (n=3 for each time point) containing 0.2 mL DMEM media for overnight before the nanoprobe incubation. 20 μg/mL ⁶⁴Cu-UPS_{6.9} or ⁶⁴Cu-PEG-PLA dispersed in either pH 6.5 or pH 7.4 DMEM medium containing 40 mg/mL human serum albumin were incubated with HN5 cells. At the specific time point, the cell wells were washed with cold PBS buffer three times to remove all the non-trapped nanoprobes. Finally, the 96-well plates were exposed on Perkin Elmer storage phosphor screens for overnight, then imaged using Typhoon imager for ⁶⁴Cu tracer quantification. For confocal imaging, after nanoprobe incubation, the cells were fixed with 4% paraformaldehyde in PBS for 10 min at RT, and permeabilized with 0.1% Triton X-100 in PBS for 10 min at 4° C. Cells were then stained by Hoechst 33342, Anti-F-Actin, and Anti-LAMP1 for nucleus, cell membrane, and lysosomes, respectively. Anti-poly(ethylene glycol) antibody was used to label the UPS_{6.9} copolymer.

G. In Vivo PET/CT Imaging

For ⁶⁴Cu-UPS_{6.9}, each mouse received ˜100 μCi of nanoprobes in 150 μL of saline intravenously via tail vein injection and PET/CT images were acquired 18-24 hours post-injection on the Siemens Inveon PET/CT Multi-Modality System for 15 mins. For FDG experiments, mice were fasted for 12 h prior to PET imaging. Each mouse received 150 μCi of FDG in 150 μL of saline intravenously via tail vein injection. PET/CT images were acquired one hour post-injection for 15 mins. The mice were sedated on the imaging bed under 2% isoflurane for the duration of imaging. Immediately after the CT data acquisition that was performed at 80 kV and 500 μA with a focal spot of 58 μm, 15-min static PET scans were conducted. The PET images were reconstructed using a Fourier Rebinning and Ordered Subsets Expectation Maximization 3D (OSEM3D) algorithm. Reconstructed CT and PET images were fused and analyzed using Inveon Research Workplace (IRW) software. PET images then were reconstructed into a single frame using the 3D Ordered Subsets Expectation Maximization (OSEM3D/MAP) algorithm. Regions of interest (ROI) were drawn manually as guided by CT encompassing the tumor in all planes containing the tissue. The target activity was quantitatively calculated as percentage injected dose per gram of tissue (% ID/g).

H. Ex Vivo Autoradiography and Histology

Immediately following PET imaging, the mice were sacrificed and tumor and major organs (e.g., the brain, liver, spleen, heart, kidney, muscle, etc.) were harvested and frozen. Section slides were prepared from each specimen. The slides were first exposed on Perkin Elmer storage phosphor screens, then imaged using Typhoon imager for ⁶⁴Cu tracer quantification, followed by fluorescence imaging using a LICOR Odyssey flatbed scanner with an 800 nm filter for ICG signal, finally H&E staining was performed for histological correlation of the tumors.

I. Statistical Analysis

Data are expressed as mean±s.d. Sample sizes were chosen to ensure adequate power (>85%, at significance of 0.05) to detect predicted effect sizes, which were estimated on the basis of either preliminary data or previous experiences with similar experiments. Differences between groups were assessed using paired, two-sided Student's t-tests for the calculation of P values.

### Prophetic Example 3: Overcoming False Positive Diagnosis as a Result of Tissue Inflammation Using ⁶⁴Cu-UPS for Determining Efficacy of Therapeutic Agents

Non-cancerous tissue inflammation (e.g., bacterial infection or tissue injury from surgery or radiation) frequently causes false positive PET results and contributes to patient worry and unnecessary testing with its attendant morbidity. Inflammatory cells use glucose as a primary source of metabolic energy, and thus increased uptake of glucose and high rates of glycolysis are characteristic of inflamed tissue (Hess et al., 2014). In this study, a lipopolysaccharide (LPS)-induced, tumor-free animal model will be set up to investigate the imaging outcome of ⁶⁴Cu-UPS at site of tissue inflammation. LPS-induced inflammation models have been widely used to study acute lung injury (de Prost et al., 2014 and Zhou et al., 2013), atherosclerosis (Rudd et al., 2010), and arthritis (Hsieh et al., 2011) by FDG-PET. In one study, LPS stimulation was shown to increase FDG uptake by 2.5-fold in macrophages (Tavakoli et al., 2013). In this study, LPS will be injected (50 μg in 20 μL of PBS; a low dose is used to avoid strong systemic inflammation) into the right hind leg muscles of C57BL/6 immunocompetent mice. At 2-4 h after injection, the serum will be collected and the pro-inflammatory cytokines will be analyzed (e.g., TNF-α and IL-10). The LPS dose will be reduced if the systemic cytokine level is high. On day 1 and 7, the animals will be imaged using FDG and ⁶⁴Cu-UPS following the protocols as previously described for tumor imaging studies. For quantitative comparison of PET images, the left hind leg without LPS injection will be used as control. The values of % ID/g and SUV will be determined. After imaging, the leg muscles will be fixed in formalin and sectioned. The density of inflammatory cells (e.g., tissue-infiltrating macrophages) will be estimated in the tissue sections and correlate it with FDG and ⁶⁴Cu-UPS signal intensity. It is anticipated that FDG will produce strong signals in inflamed tissues due to the high rate of glucose uptake in infiltrating inflammatory cells. In contrast, it is anticipated that the ⁶⁴Cu-UPS signal will be low due to the fast clearance of protons by the healthy lymphatic system and limited nanoprobe extravasation at inflamed sites (unlike tumors with leakier vasculature and damaged lymphatics, aka extended permeation and retention effect; Fang et al., 2011 and Maeda et al., 2000).

It is possible that tissue inflammation can activate ⁶⁴Cu-UPS through the high glycolysis rates of the inflammatory cells. If persistence of ⁶⁴Cu-UPS signals in the LPS-injected site is observed, investigation of administration of taurine chloramine (TauCl) will be commenced, which has been shown to abrogate the FDG signals in macrophages after LPS stimulation (Kim et al., 2009). TauCl is generated and released from activated neutrophils following apoptosis, which exerts anti-inflammatory properties by inhibiting the production of inflammatory mediators (e.g., TNF-α; Kim et al., 2014 and Marcinkiewicz et al. 2014). TauCl will be tested to evaluate whether it can specifically decrease ⁶⁴Cu-UPS signals in inflammatory cells.

### Prophetic Example 4: Monitoring Efficacy of Other Therapies Using ⁶⁴C-UPS Probes as a Noninvasive Tool

In recent years, FDG-PET has been increasingly used to monitor therapeutic response in patients undergoing radiation or chemo-radiation therapy (Challapalli et al. 2016 and Weber, 2005). A notable clinical pain point for FDG-PET is the false positives arising from therapy (e.g., radiation-induced tissue inflammation), or false negatives from shrinking tumors after treatment. It is hypothesized that ⁶⁴Cu-UPS-PET offers an accurate imaging method to monitor the antitumor efficacy of radiation and/or chemotherapy in selected head and neck cancer models. Feasibility of ⁶⁴Cu-UPS-PET to predict the antitumor efficacy of small molecular inhibitors targeting acidosis pathways will be investigated. If successfully demonstrated, ⁶⁴Cu-UPS-PET has the potential to predict tumor response to existing therapies including, but limited to, chemo-radiation therapy or novel small molecular tumor acidosis inhibitors, at an early stage in the course of therapy, thereby reducing the side effects and cost of ineffective therapy.

Various head and neck tumors may have different sensitivities toward chemo-radiation therapy. Depending on the ⁶⁴Cu-UPS-PET response, the intensity of the therapy will be either increased or decreased and its effect on antitumor efficacy and treatment morbidity investigated. On the other hand, if chemo-radiation induces high levels of false positives/negatives in ⁶⁴Cu-UPS-PET diagnosis, the mechanism will be identified, and mitigation strategy developed to minimize the false rates.

All of the compositions and methods disclosed and claimed herein can be made and executed without undue experimentation in light of the present disclosure. While the compositions and methods of this disclosure have been described in terms of certain embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### K. References

The following references, to the extent that they provide exemplary procedural or other details supplementary to those set forth herein, are specifically incorporated herein by reference.
WO 2013/152059
WO 2015/188157
Adams and Weiner, Nat. Biotechnol., 23:1147-1157, 2005.
Albertazzi et al., J. Am. Chem. Soc. 2010, 132, 18158.
Alberts et al., Molecular Biology of the Cell; 5th ed.; Garland Science: New York, 2008.
Almutairi et al., J. Am. Chem. Soc. 2007, 130, 444.
Ananthapadmanabhan et al., Langmuir 1985, 1, 352.
Atkins and De Paula, Physical Chemistry; Oxford University Press, 2009.
Atkins et al., J. Surg. Res., 177, 109-115, 2012.
Benjaminsen et al., ACS Nano 2011, 5, 5864.
Berezin and Achilefu, Chem. Rev. 2010, 110, 2641.
Bhargava et al., Clin. Nucl. Med., 36, e20-29, 2011.
Blanco et al., Cancer Res., 70, 3896-3904, 2010.
Blodgett et al., Clin. Imaging, 35, 49-63, 2011.
Blum et al., Nat. Chem. Biol. 2005, 1, 203.
Bolte and Cordelieres, J. Microsc. 224, 213-232, 2006.
Cardone et al., Nat. Rev. Cancer, 5, 786-795, 2005.
Casey et al., Nat. Rev. Mol. Cell Biol., 11:50-61, 2010.
Castaigne et al., In: Head and Neck Cancer Imaging (ed R. Hermans) 329-343, Springer, 2006.
Challapalli and Aboagye, Front. Oncol., 2016, 6, 00044.
Choi et al., Nat. Biotechnol. 31, 148-153, 2013.
Christofk et al., Nature, 452:230-233, 2008.
Cohade et al., J. Nucl. Med., 44, 1267-1270, 2003.
Collins and Washabaugh, Q. Rev. Biophys. 18:323-422, 1985.
Collins, Biophys. J. 1997, 72, 65-76.
Conner and Schmid, Nature, 422:37-44, 2003.
Cook et al., Semin. Nucl. Med. 34, 122-133, 2004.
Cross and Muller, FEBS Lett., 576:1-4, 2004.
Culverwell, Clin. Radiol., 66, 366-382, 2011.
Dacosta et al., Best Pract. Res. Clin. Gastroenterol., 20, 41-57, 2006.
Dai et al., Soft Matter, 2008, 4, 435.
Dale and Rebek, J. Am. Chem. Soc. 2006, 128, 4500.
de Prost et al., J. Nucl. Med., 2014, 55, 1871-1877.
de Silva et al., Chem. Commun. 1996, 2399.
de Silva et al., Chem. Rev. 1997, 97, 1515.
Deamer et al., Membrane permeability: 100 years since Ernest Overton, Academic Press, San Diego, CA, USA; 1999.
Demaurex, News Physiol. Sci. 2002, 17, 1-5.
Demchenko, Introduction to Fluorescence Sensing; Springer Science: New York, 2008.
Diaz-Fernandez et al., Chemistry, 12, 921-930, 2006.
Diaz-Fernandez, Chem. Eur. J. 2006, 12, 921.
Diwu et al., Chem. Biol., 6: 411-418, 1999.
Draga et al., Anal. Chem., 82, 5993-5999, 2010.
Eigenbrodt et al., Anticancer Res., 18:3267-3274, 1998.
Enerson and Drewes, J. Pharm. Sci., 92:1531-1544, 2003.
Fang et al., Adv. Drug Deliv. Rev., 2011, 63, 136-151.
Fantin et al., Cancer Cell, 9:425-434, 2006.
Fernandez-Suarez and Ting, Nat. Rev. Mol. Cell Biol. 2008, 9, 929.
Fink et al., J. Nucl. Med. 56, 1554-1561, 2015.
Folkman, Nat. Rev. Drug Discovery, 2007, 6, 273-286.
Fukui et al., Radiographics, 25, 913-930, 2005.
Gallagher et al., Nature, 453, 940-943, 2008.
Gatenby and Gillies, Nat. Rev. Cancer, 2004, 4, 891-899.
Gatenby and Gillies, Nat. Rev. Cancer, 2008, 8, 56-61.
Giepmans et al., Prog. Polym. Sci. 2004, 29, 1173.
Gillies et al., Am. J. Physiol., 267, C195-C203, 1994.
Gillies et al., Med. Biol. Mag., 23, 57-64, 2004.
Gould et al., JAMA, 285, 914-924, (2001).
Gross and Piwnica-Worms, Cancer Cell, 2005, 7, 5.
Grunwell et al., J. Am. Chem. Soc. 2001, 123, 4295-4303.
Ha et al., Proc. Natl. Acad. Sci. U.S.A 1999, 96, 893-898
Haka et al., Cancer Res., 66, 3317-3322, 2006.
Halestrap and Price, Biochem. J., 343 Pt 2, 281-299, 1999.
Han and Burgess, Chem. Rev. 2010, 110, 2709.
Hanahan and Weinberg, Cell, 2011, 144, 646-674.
Harvey et al., Head Neck, 32, 76-84, 2010.
Haubner and Wester, Curr. Pharm. Des., 10:1439-1455, 2004.
Hawkins and Davis, Pharmacol. Rev., 57:173-185, 2005.
Heiden et al., Science, 324, 1029-1033, 2009.
Heiden, Science, 324, 1029-1033, 2009.
Hensley et al., Cell, 164, 681-694, 2016.
Hess et al., PET Clin., 2014, 9, 497-519.
Hofmeister, Pathol. u. Pharmakol, 25, 1888.
Holtzman, Lysosomes, Springer, New York, NY, USA, 1989.
Hsieh et al., J. Ethnopharmacol., 2011, 136, 511-517.
Huang et al., ACS Nano, 2010, 4, 5887-5896.
Huotari and Helenius, EMBO J, 30:3481-3500, 2011.
Iczkowski and Lucia, Prostate Cancer, 2011, 673021, 2011.
Imamura et al., Proc. Natl. Acad. Sci. U.S.A, 100: 2312-2315, 2003.
Izumi et al., Cancer Treat. Rev. 2003, 29, 541.
Jacobs et al., Am. J. Clin. Pathol. 113, 251-258, 2000.
Jares-Erijman and Jovin, Nat. Biotech., 2003, 21, 1387-1395.
Jelley, Nature, 1936, 138, 1009.
Johansson and Cook, Chem. Eur. J., 2003, 9, 3466.
Jose et al., Biochim. Biophys. Acta, 1807:552-561, 2011.
Joyce, Cancer Cell, 2005, 7, 513-520.
Kairdolf and Nie, J. Am. Chem. Soc. 2011, 133, 7268.
Kalyanasundaram and Thomas, J. Am. Chem. Soc., 99:2039-2044, 1977.
Kanter et al., Am. J. Obstet. Gynecol., 200, 512 e511-515, 2009.
Kim and Cha, Amino Acids, 2014, 46, 89-100.
Kim and Kim, Adv. Exp. Med. Biol. 2009, 643, 473-480.
Kim et al., Cell, 155:552-566, 2013.
Kleiter et al., Int. J. Radiat. Oncol. Biol. Phys., 2006, 64, 592-602.
Kobayashi and Choyke, Acc. Chem. Res. 2010, 44, 83.
Kobayashi et al., Chem. Rev. 2010, 110, 2620.
Kreuter, Adv. Drug Deliv. Rev., 47:65-81, 2001.
Kubben et al., Lancet Oncol., 12, 1062-1070, 2011.
Kunz et al., Curr. Opin. Colloid Interface Sci. 2004, 9, 1-18.
Lakowicz, Principles of Fluorescence Spectroscopy; 3rd ed.; Springer: New York City, 2006.
Lee et al., Chem. Commun. 2008, 4250.
Lee et al., Curr. Top. Med. Chem. 2010, 10, 1135.
Lee et al., J. Control. Release, 2003, 90, 363.
Lee et al., Nano Lett. 2009, 9, 4412.
Leontidis, Curr. Opin. Colloid Interface Sci. 2002, 7, 81-91.
Levi et al., J. Am. Chem. Soc. 2010, 132, 11264.
Li et al., Adv. Funct. Mater. 2010, 20, 2222.
Li et al., Angew. Chem. Int. Ed., 53:8074-8078, 2014.
Li et al., Chem. Rev. 118, 5359-5391, 2018.
Liu et al., ACS Nano, 4, 2755-2765, 2010.
Liu et al., Angew. Chem. Int. Ed., 48:7346-7349, 2009.
Lodder et al., Eur. Radiol., 21, 98-106, 2011.
López Arbeloa and Ruiz Ojeda, Chem. Phys. Lett. 1982, 87, 556.
Lopez-Fontal et al., Chem. Sci., 7, 4468-4475, 2016.
Lou et al., Cancer Res. 71, 3364-3376, 2011.
Lovell et al., Chem. Rev. 2010, 110, 2839.
Ma et al., J. Am. Chem. Soc., 136:11085-11092, 2014.
Ma et al., Macromolecules 2003, 36, 3475.
Maeda et al., J. Control. Release, 2000, 65, 271-284.
Manders et al., J. Microsc., 169: 375-382, 1993.
Marcinkiewicz and Kontny, Amino Acids, 2014, 46, 7-20.
Maxfield and McGraw, Nat. Rev. Mol. Cell Biol., 5, 121-132, 2004.
Maxwell et al., Bioconjug. Chem. 2009, 20, 702.
McElroy et al., World J. Surg., 32, 1057-1066, 2008.
McMahon et al., Br. J. Oral Maxillofac. Surg., 41, 224-231, 2003.
Merk et al., Langmuir 2014, 30, 4213-4222.
Mirbolooki, EJNMMI Research, 1:30, 2011.
Mo et al., Anal. Chem., 81, 8908-8915, 2009.
Modi et al., Nat. Nanotech. 2009, 4, 325.
Moghimi et al., Prog. Lipid Res. 42, 463-478, 2003.
Morgan, Mol. Membr. Biol., 21:423-433, 2004.
Nasongkla et al., Nano. Lett. 2006, 6, 2427-2430.
Neri and Supuran, Nat. Rev. Drug Discov. 10, 767-777, 2011.
Nguyen and Tsien, Nat. Rev. Cancer, 13, 653-662, 2013.
Nishi and Forgac, Nat. Rev. Mol. Cell Biol. 2002, 3, 94.
Ogawa et al., ACS Chem. Biol. 2009, 4, 535.
Ohgaki et al., Biochemistry 2010, 50, 443.
Omuro and DeAngelis, JAMA, 310, 1842-1850, 2013.
Pacchiano et al., J. Med. Chem., 54, 1896-1902, 2011.
Packard et al. J. Phys. Chem. B 1997, 101, 5070.
Paik et al., J. Natl. Cancer Inst., 92, 1991-1998, 2000.
Parks et al., Nat. Rev. Cancer, 13, 611-623, 2013.
Parsons et al., Phys. Chem. Chem. Phys. 2011, 13, 12352-12367.
Pena-Llopis et al., EMBO J., 30: 3242-3258, 2011.
Perez-Sayans et al., Cancer Treat. Rev., 35:707-713, 2009.
Perkins et al., Nucl. Med. Commun. 34, 168-174, 2013.
Petsalakis et al., J. Mol. Struct.: THEOCHEM, 2008, 867, 64.
Pouyssegur et al., Nature, 441:437-443, 2006.
Purohit et al., Insights Imaging, 5, 585-602, 2014.
Ramanujam et al., Photochem. Photobiol., 64, 720-735, 1996.
Ramirez et al., Cancer Res, 64:9027-9034, 2004.
Ravasz et al., J. Craniomaxillofac. Surg., 19, 314-318, 1991.
Riess, Prog. Polym. Sci., 2003, 28, 1107.
Roczniak-Ferguson et al., Sci. Signal, 5, ra42, 2012.
Rodman et al., Exp. Cell Res., 192:445-452, 1991.
Rossin et al., J. Nucl. Med., 49:103-111, 2008.
Ruckenstein and Nagarajan, J. Phys. Chem. 1975, 79, 2622.
Rudd et al., J. Am. Coll. Cardiol., 2010, 55, 2527-2535.
Sancak et al., Cell, 141: 290-303, 2010.
Sapsford et al., Angew. Chem. Int. Ed. 2006, 45, 4562-4589.
Scheibe, Angew. Chem. 1936, 49, 563.
Schmalfuss, In: Head and Neck Cancer Imaging (ed I. Hermans) 363-385, Springer, 2012.
Schoder, In: Nuclear oncology: pathophysiology and clinical applications (eds H. W. Strauss, G. Mariani, D. Volterrani, & S. M. Larson) 269-295, Springer, 2013.
Schomacker et al., Lasers Surg. Med., 12, 63-78, 1992.
Schwarz et al., Cancer, 115, 1669-1679, 2009.
Settembre et al., EMBO J., 31:1095-1108, 2012.
Settembre et al., Nat. Rev. Mol. Cell Biol., 14:283-296, 2013.
Som, P. et al. J. Nucl. Med., 21, 670-675, 1980.
Sonveaux et al., J. Clin. Invest., 118, 3930-3942, 2008.
Srikun et al., Chem. Sci. 2011, 2, 1156.
Sterling et al., Cell Physiol., 283:C1522-1529, 2002.
Suh et al., Bioorg. Chem., 22: 318-327, 1994.
Sun et al., J. Med. Chem., 45:469-477, 2002.
Supuran, Bioorg. Med. Chem. Lett., 20:3467-3474, 2010.
Supuran, Nat. Rev. Drug Discov., 7:168-181, 2008.
Swartz et al., Cancer Res., 2012, 72, 2473-2480.
Tal et al., Chem. Eur. J., 2006, 12, 4858.
Tavakoli et al., J. Nucl. Med., 2013, 54, 1661-1667.
Tran et al., Hepatogastroenterology, 59, 1994-1999, 2012.
Truong et al., J. Comput. Assist. Tomogr., 29, 205-209, 2005.
Truong et al., Radiol. Clin. North Am., 52, 17-25, 2014.
Tsarevsky and Matyjaszewski, Chem. Rev. 2007, 107, 2270-2299.
Tsien, Nat. Rev. Mol. Cell Biol. 2003, 4, SS16.
Ueno and Nagano, Nat. Methods, 2011, 8, 642.
Underwood and Anacker, J. Colloid Interface Sci., 1987, 117, 242-250.
Urano et al., Nat. Med., 15:104-109, 2008.
Vahrmeijer et al., Nat. Rev. Clin. Oncol., 10, 507-518, 2013.
Valdes-Aguilera and Neckers, Acc. Chem. Res., 1989, 22, 171.
Valeur, Molecular fluorescence: principles and applications; Wiley-VCH, 2002.
van Sluis et al., Magn. Reson. Med., 41, 743-750, 1999.
Vishvakarma and Singh, Biomed. Pharmacother. 65, 27-39, 2011.
Vogel et al., Sci. STKE 2006, 2006, re2.
Vogelstein et al., Science, 339, 1546-1558, 2013.
Volk et al., Br. J. Cancer, 68, 492-500, 1993.
Wadas et al., Curr. Pharm. Des., 13:3-16, 2007.
Wang et al., Angew. Chem. Int. Ed. 2008, 47, 9726-9729.
Wang et al., J. Vis. Exp., 2012.
Wang et al., Nat. Mater., 13:204-212, 2014.
Wasielewski, Chem. Rev. 1992, 92, 435.
Webb et al., Nat. Rev. Cancer, 11, 671-677, 2011.
Weber et al., J. Nucl. Med., 2005, 46, 983-995.
Weerakkody et al., Proc. Natl. Acad. Sci. U.S.A, 110, 5834-5839, 2013.
Weis and Cheresh, Nat. Med., 2011, 17, 1359-1370.
Weissleder and Pittet, Nature 2008, 452, 580.
Weisz, Cytol., 226:259-319, 2003.
Weller, Pure Appl. Chem. 1968, 16, 115.
Wen and Kesari, N. Engl. J. Med., 359, 492-507, 2008.
West and Pearce, J. Phys. Chem., 1965, 69, 1894.
Williamson, Nat. Chem. Biol., 4, 458-465, 2008.
Winnik, Chem. Rev., 93:587-614, 1993.
Woodin et al., Eur. J. Inorg. Chem., 4829-4833, 2005.
Woolgar and Triantafyllou, Oral Oncol., 41, 1034-1043, 2005.
Yang et al., Cancer Res., 2009, 69, 7986-93.
Yeung et al., Curr. Opin. Cell Biol., 18, 429-437, 2006.
Yezhelyev et al., J. Am. Chem. Soc., 2008, 130, 9006.
Yu et al., ACS Nano, 2011, 5, 9246.
Zhang and Cremer, Curr. Opin. Chem. Biol. 2006, 10, 658-663.
Zhang and Cremer, Proc. Natl. Acad. Sci. U.S.A 2009, 106, 15249-15253.
Zhang et al., Cell Metab. 20:526-540, 2014.
Zhang et al., Nat. Rev. Mol. Cell Biol. 2002, 3, 906.
Zhang et al., Nucl. Med. 2010, 51, 1167.
Zhao et al., Nat. Biomed. Eng., 1, 2016.
Zhou et al., Exp. Ther. Med., 2013, 6, 894-898.
Zhou et al., J. Am. Chem. Soc., 134:7803-7811, 2012.
Zhou et al., Macromolecules, 2008, 41, 8927.
Zhou et. al., Angew. Chem. Int. Ed., 2011, 50, 6109-6114.
Zhu et al., Semin. Oncol. 38, 55-69, 2011.
Zoncu et al., Science, 334:678-683, 2011.

1. A polymer of the formula: 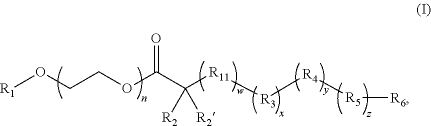
wherein:
R₁ is hydrogen, alkyl_{(C≤12)}, cycloalkyl_{(C≤12)}, substituted alkyl_{(C≤12)}, substituted cycloalkyl_{(C≤12)}, or 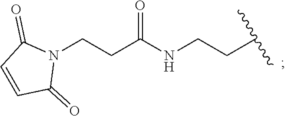 [SMILES: *CCNC(=O)CCN1C(=O)C=CC1=O]
n is an integer from 1 to 500;
R₂ and R₂′ are each independently selected from hydrogen, alkyl_{(C≤12)}, cycloalkyl_{(C≤12)}, substituted alkyl_{(C≤12)}, or substituted cycloalkyl_{(C≤12)};
R₃ and R₁₁ are each independently a group of the formula: 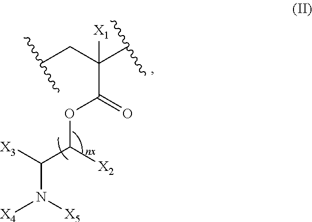
wherein:
nₓ is 1-10;
X₁, X₂, and X₃ are each independently selected from hydrogen, alkyl_{(C≤12)}, cycloalkyl_{(C≤12)}, substituted alkyl_{(C≤12)}, or substituted cycloalkyl_{(C≤12)}; and
X₄ and X₅ are each independently selected from alkyl_{(C≤12)}, cycloalkyl_{(C≤12)}, aryl_{(C≤12)}, heteroaryl_{(C≤12)} or a substituted version of any of these groups, or X₄ and X₅ are taken together and are alkanediyl_{(C≤12)}, alkoxydiyl_{(C≤12)}, alkylaminodiyl_{(C≤12)}, or a substituted version of any of these groups;
w is an integer from 0 to 150;
x is an integer from 1 to 150;
R₄ is a group of the formula: 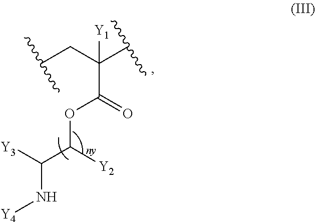
wherein:
n_{y} is 1-10;
Y₁, Y₂, and Y₃ are each independently selected from hydrogen, alkyl_{(C≤12)}, cycloalkyl_{(C≤12)}, substituted alkyl_{(C≤12)}, or substituted cycloalkyl_{(C≤12)}; and
Y₄ is indocyanine green (ICG);
y is an integer from 1-6;
R₅ is a group of the formula: 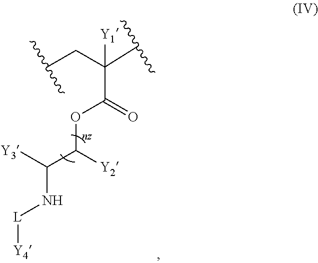
wherein:
n_{z} is 1-10;
Y₁′, Y₂′, and Y₃′ are each independently selected from hydrogen, alkyl_{(C≤12)}, cycloalkyl_{(C≤12)}, substituted alkyl_{(C≤12)}, or substituted cycloalkyl_{(C≤12)}; and
Y₄′ is a metal chelating group;
L is a covalent bond; or
alkanediyl_{(C≤12)}, arenediyl_{(C≤12)}, -alkanediyl_{(C≤12)}-arenediyl_{(C≤12)}—NC(S)—, -alkanediyl_{(C≤12)}-arenediyl_{(C≤12)}-C(O)—, or a substituted version of any of these groups;
z is an integer from 1-6; and
R₆ is hydrogen, halo, hydroxy, alkyl_{(C≤12)}, or substituted alkyl_{(C≤12)},
wherein R₁₁, R₃, R₄, and R₅ can occur in any order within the polymer.

2. The polymer of claim 1, further defined by the formula wherein:
R₁ is hydrogen, alkyl_{(C≤12)}, substituted alkyl_{(C≤12)}, or 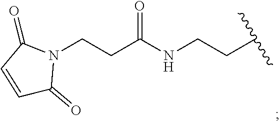 [SMILES: *CCNC(=O)CCN1C(=O)C=CC1=O]
n is an integer from 10 to 500;
R₂ and R₂′ are each independently selected from hydrogen, alkyl_{(C≤12)}, or substituted alkyl_{(C≤12)};
R₃ and R₁₁ are each independently a group of the formula: 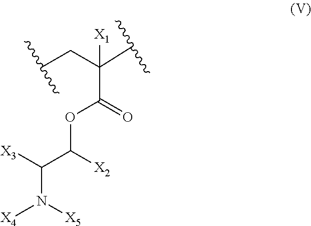
wherein:
X₁, X₂, and X₃ are each independently selected from hydrogen, alkyl_{(C≤12)}, or substituted alkyl_{(C≤12)}; and
X₄ and X₅ are each independently selected from alkyl_{(C≤12)}, aryl_{(C≤12)}, heteroaryl_{(C≤12)} or a substituted version of any of these groups, or X₄ and X₅ are taken together and are alkanediyl_{(C≤8)}, alkoxydiyl_{(C≤8)}, alkylaminodiyl_{(C≤8)}, or a substituted version of any of these groups;
x is an integer from 1 to 100;
w is an integer from 0 to 100;
R₄ is a group of the formula: 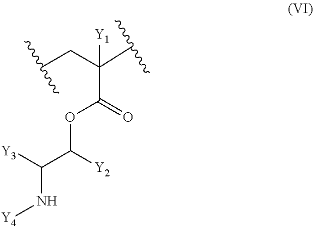
wherein:
Y₁, Y₂, and Y₃ are each independently selected from hydrogen, alkyl_{(C≤12)}, or substituted alkyl_{(C≤12)}; and
Y₄ is indocyanine green (ICG);
y is an integer from 1 to 6;
R₅ is a group of the formula: 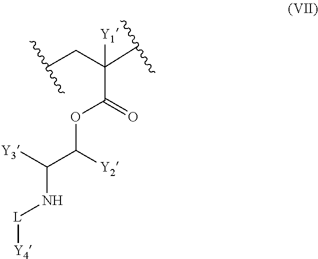
wherein:
Y₁′, Y₂′, and Y₃′ are each independently selected from hydrogen, alkyl_{(C≤12)}, substituted alkyl_{(C≤12)}; and
Y₄′ is a metal chelating group;
L is a covalent bond; or
alkanediyl_{(C≤12)}, arenediyl_{(C≤12)}, -alkanediyl_{(C≤12)}-arenediyl_{(C≤12)}—NC(S)—, -alkanediyl_{(C≤12)}-arenediyl_{(C≤12)}-C(O)—, or a substituted version of any of these groups;
z is an integer from 1-6; and
R₆ is hydrogen, halo, alkyl_{(C≤12)}, or substituted alkyl_{(C≤12)},
wherein R₁₁, R₃, R₄, and R₅ can occur in any order within the polymer.

3. The polymer of claim 1, further defined by the formula wherein:
R₁ is hydrogen, alkyl_{(C≤8)}, substituted alkyl_{(C≤8)}, or 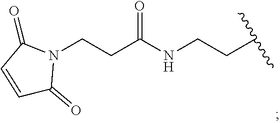 [SMILES: *CCNC(=O)CCN1C(=O)C=CC1=O]
n is an integer from 10 to 200;
R₂ and R₂′ are each independently selected from hydrogen, alkyl_{(C≤8)}, or substituted alkyl_{(C≤8)};
R₃ and R₁₁ are each independently a group of the formula: 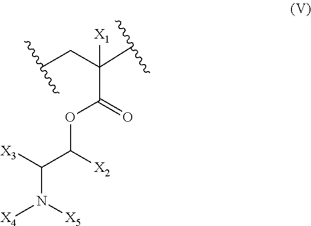
wherein:
X₁, X₂, and X₃ are each independently selected from hydrogen, alkyl_{(C≤8)}, or substituted alkyl_{(C≤8)}; and
X₄ and X₅ are each independently selected from alkyl_{(C≤12)}, aryl_{(C≤12)}, heteroaryl_{(C≤12)} or a substituted version of any of these groups, or X₄ and X₅ are taken together and are alkanediyl_{(C≤8)} or substituted alkanediyl_{(C≤8)};
x is an integer from 1 to 100;
w is an integer from 0 to 100;
R₄ is a group of the formula: 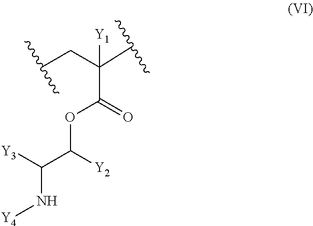
wherein:
Y₁, Y₂, and Y₃ are each independently selected from hydrogen, alkyl_{(C≤8)}, or substituted alkyl_{(C≤8)}; and
Y₄ is indocyanine green (ICG);
y is an integer from 1 to 6;
R₅ is a group of the formula: 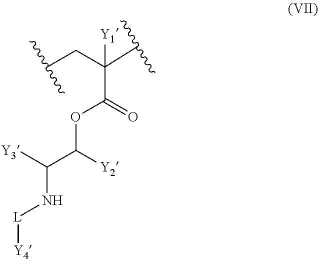
wherein:
Y₁′, Y₂′, and Y₃′ are each independently selected from hydrogen, alkyl_{(C≤8)}, substituted alkyl_{(C≤8)}; and
Y₄′ is a metal chelating group;
L is a covalent bond; or
alkanediyl_{(C≤12)}, arenediyl_{(C≤12)}, -alkanediyl_{(C≤12)}-arenediyl_{(C≤12)}—NC(S)—, -alkanediyl_{(C≤12)}-arenediyl_{(C≤12)}-C(O)—, or a substituted version of any of these groups;
z is an integer from 1-6; and
R₆ is hydrogen, halo, alkyl_{(C≤6)}, or substituted alkyl_{(C≤6)},
wherein R₁₁, R₃, R₄, and R₅ can occur in any order within the polymer.

4. The polymer of claim 1, wherein R₁ is hydrogen.

5. The polymer of claim 1, wherein R₁ is alkyl_{(C≤6)}.

6. (canceled)

7. The polymer of claim 1, wherein R₁ is

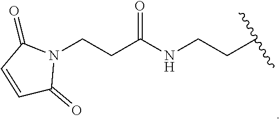 [SMILES: *CCNC(=O)CCN1C(=O)C=CC1=O]

8. The polymer of claim 1, wherein R₂ is alkyl_{(C≤6)}.

9. (canceled)

10. The polymer of claim 1, wherein R₂′ is alkyl_{(C≤6)}.

11. (canceled)

12. The polymer of claim 1, wherein R₃ or R₁₁ is further defined by the formula: 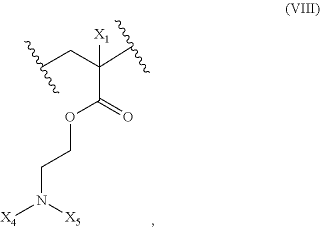
wherein:
X₁ is selected from hydrogen, alkyl_{(C≤8)}, or substituted alkyl_{(C≤8)}; and
X₄ and X₅ are each independently selected from alkyl_{(C≤12)}, aryl_{(C≤12)}, heteroaryl_{(C≤12)} or a substituted version of any of these groups, or X₄ and X₅ are taken together and are alkanediyl_{(C≤8)} or substituted alkanediyl_{(C≤8)}.

13.-22. (canceled)

23. The polymer of claim 1, wherein R₄ is further defined by the formula: 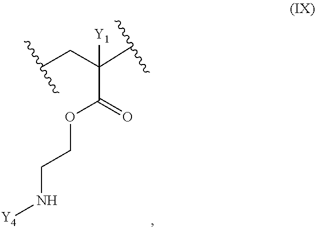
wherein:
Y₁ is selected from hydrogen, alkyl_{(C≤8)}, or substituted alkyl_{(C≤8)}; and
Y₄ is indocyanine green (ICG).

24.-32. (canceled)

33. The polymer of claim 1, wherein each R₁₁ is incorporated consecutively to form a block.

34. The polymer of claim 1, wherein each R₃ is incorporated consecutively to form a block.

35. The polymer of claim 1, wherein each R₁₁ is present as a block and each R₃ is present as a block.

36. The polymer of claim 1, wherein each R₁₁ and each R₃ are randomly incorporated within the polymer.

37. The polymer of claim 1, wherein R₅ is further defined by the formula: 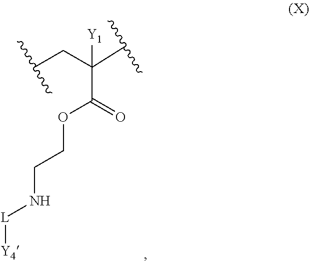
wherein:
Y₁′ is selected from hydrogen, alkyl_{(C≤8)}, substituted alkyl_{(C≤8)};
Y₄′ is a metal chelating group; and
L is a covalent bond; or
alkanediyl_{(C≤12)}, arenediyl_{(C≤12)}, -alkanediyl_{(C≤12)}-arenediyl_{(C≤12)}—NC(S)—, -alkanediyl_{(C≤12)}-arenediyl_{(C≤12)}-C(O)—, or a substituted version of any of these groups.

38.-58. (canceled)

59. The polymer of claim 1, wherein n is 75-150.

60. (canceled)

61. The polymer of claim 1, wherein x is 1-99.

62. The polymer of claim 61, wherein x is from 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-199 or any range derivable therein.

63. The polymer of claim 1, wherein y is 1, 2, 3, 4, or 5.

64.-65. (canceled)

66. The polymer of claim 1, wherein z is 1, 2, 3, 4, or 5.

67.-68. (canceled)

69. The polymer of claim 1, wherein each R₁₁, R₃, R₄, and R₅ can occur in any order within the polymer.

70. The polymer of claim 1, wherein each R₁₁, R₃, R₄, and R₅ occur in the order described in formula I.

71. The polymer of claim 1, wherein w is 0.

72. The polymer of claim 1, wherein the polymer further comprises a targeting moiety.

73. (canceled)

74. The polymer of claim 1, wherein R₃ and R₁₁ are selected from:

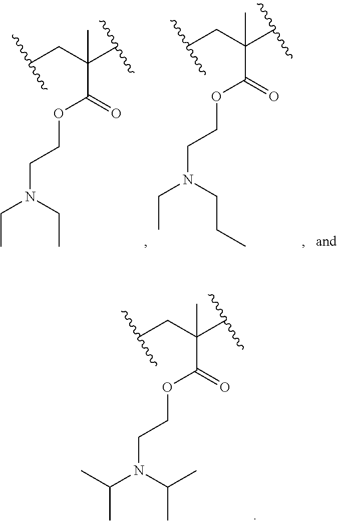 [SMILES: *CC(*)(C)C(=O)OCCN(CC)CC ; *CC(*)(C)C(=O)OCCN(CC)CCC ; *CC(*)(C)C(=O)OCCN(C(C)C)C(C)C]

75. The polymer of claim 1, wherein R₃ is:

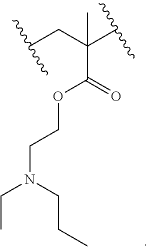 [SMILES: *CC(*)(C)C(=O)OCCN(CC)CCC]

77. The polymer of claim 1, wherein the polymer is UPS_{6.9}.

78. A micelle of a polymer of claim 1.

79. A pH responsive system comprising a micelle of a first polymer wherein the first polymer has a formula claim 1, wherein Y₄ is indocyanine green (ICG), and wherein the micelle has a pH transition point and an emission spectra.

80.-93. (canceled)

94. A method of imaging the pH of an intracellular or extracellular environment comprising:
(a) contacting a pH responsive system of claim 79 with the environment; and
(b) detecting one or more signals from the environment, wherein the detection of the signal indicates that the micelle has reached its pH transition point and disassociated.

95.-111. (canceled)

112. A method of delivering a compound of interest to a target cell comprising:
(a) encapsulating the compound of interest with a pH responsive system of a polymer of claim 1; and
(b) contacting the target cell with the pH responsive system under such conditions that the pH of the target cell triggers the disassociation of the pH responsive system and release of the compound, thereby delivering the compound of interest.

113.-116. (canceled)

117. A method of resecting a tumor in a patient comprising:
(a) administering to the patient an effective dose of a pH responsive system of claim 79;
(b) detecting one or more signals for the patient; wherein the one of more signals indicate the presence of a tumor; and
(c) resecting the tumor via surgery.

118.-128. (canceled)

129. A method of treating a cancer susceptible to endosomal/lysosomal pH arrest in a patient comprising administering to the patient in need thereof a pH responsive system of claim 79.

130.-132. (canceled)

133. A method of identifying the tumor acidosis pathway comprising:
(a) contacting a pH responsive system comprising one or more micelles of claim 79 with a cell or a cellular environment;
(b) contacting the cell with an inhibitor of the pH regulatory pathway;
(c) detecting a signal from the cell or cellular environment, wherein the detection of the signal indicates that one of the micelles has reached its pH transition point and disassociated; and
(d) correlating the signal with a modification in the tumor acidosis pathway.

134.-139. (canceled)

140. A method of imaging a patient to determine the presence of a tumor comprising:
(a) contacting a pH responsive system comprising one or more micelles of claim 79 with the tumor;
(b) collecting one or more PET or SPECT imaging scans; and
(c) collecting one or more optical imaging scans, wherein the detection of the optical signal indicates that one of the micelles has reached its pH transition point and disassociated;
wherein the one or more PET or SPECT imaging scans and the one or more optical imaging scans result in the identification of a tumor.

141.-149. (canceled)

150. A method of determining the efficacy of a cancer treatment therapy comprising:
(a) administering a pH responsive system comprising one or more micelles of claim 79 to a patient, wherein the patient has a tumor;
(b) collecting one or more PET or SPECT imaging scans;
(c) collecting one or more optical imaging scans, wherein the detection of the optical signal indicates that one of the micelles has reached its pH transition point and disassociated;
(d) administering the cancer treatment therapy;
(e) repeating steps (a)-(c) to determine the efficacy of the cancer treatment therapy.

151.-152. (canceled)

153. A method of treating a disease or disorder in a patient in need thereof comprising administering to the patient a polymer of claim 1.

154.-158. (canceled)
